(19) United States
(12) Patent Application Publication
(10) Pub. No.: US 2007/0015154 A1
(43) Pub. Date: Jan. 18, 2007

(54) Differentially expressed nucleic acids that correlate with ksp expression

(75) Inventors: Priti S. Hedge, Collegeville, PA (US); Pearl S. Huang, Collegeville, PA (US); Jeffrey R. Jackson, Collegeville, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: SMITHKLINE BEECHAM CORPORATION, Philidelphia, PA (US)

(21) Appl. No.: 10/544,704

(22) PCT Filed: Feb. 13, 2004
(86) PCT No.: PCT/US04/04276

§ 371 Date: May 26, 2006

Related U.S. Application Data
(60) Provisional application No. 60/447,842, filed on Feb. 14, 2003.

(51) Int. Cl. C12Q 1/68

(52) U.S. Cl. 435/6

(57) ABSTRACT

Nucleic acids that differentially expressed in certain tumors are provided. A variety of classification, screening, diagnostic and treatment methods are provided based upon these differentially expressed nucleic acids. Devices and kits for performing such methods are also disclosed.

### CROSS-REFERENCE TO RELATED APPLICATIONS

This application claims the benefit of U.S. Provisional Application No. 60/447,842, filed Feb. 14, 2003, which is incorporated herein by reference in its entirety.

### BACKGROUND

The mitotic spindle has long been an important functional target in cancer chemotherapy. This is because the mitotic spindle, composed primarily of microtubules, is responsible for the distribution of replicate copies of the genome to each of the two daughter cells that result from cell division. It is presumed that it is the disruption of the mitotic spindle by chemotherapeutics that results in inhibition of cancer cell division. This in turn results in cancer cell death. The importance of the mitotic spindle as a target is evidenced by the clinical and commercial success of the anti-tubulin agents vincristine, vinblastine and vinorelbine (Vinca alkaloids), as well as the taxanes, paclitaxel and docetaxel. All these therapeutics target tubulin, the building block for microtubules.

The problem with targeting the mitotic spindle, however, is that the microtubules that make up the spindle play critical roles in non-proliferating terminally differentiated cells in addition to their role during the interphase portion of the cell cycle. Microtubules, for example, play an essential role in neuronal transport. Neurotoxicity has terminated the development of several tubulin binding drugs and is also a significant side-effect of pacilitaxel, dotexel and vincristine. So therapeutics targeting tubulin can have side effects that limit their usefulness.

These difficulties have prompted efforts to identify chemotherapeutic agents having a different anti-mitotic mechanism. One approach has been to inhibit kinesin motor proteins. The advantage of such an approach is that these proteins have no role outside of mitosis. Inhibitors of kinesins thus would not be expected to cause the undesirable side effects associated with tubulin binding compounds.

Mitotic kinesins are enzymes essential for assembly and function of the mitotic spindle, but are not generally part of other microtubule structures, such as in nerve processes. Mitotic kinesins play essential roles during all phases of mitosis. These enzymes are “molecular motors” that transform energy released by hydrolysis of ATP into a mechanical force which drives the directional movement of cellular cargoes along microtubules. The catalytic domain sufficient for this task is a compact structure of approximately 350 amino acids. During mitosis, kinesins organize microtubules into the bipolar structure that is the mitotic spindle and slide the microtubules relative to one another, thus forcing the two spindle poles apart. Kinesins also mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death.

One of the mitotic kinesins that have been identified is KSP (Kinesin-like 1, also termed HsEgS). KSP belongs to an evolutionarily conserved kinesin subfamily of plus end-directed microtubule motors that assemble into bipolar homotetramers consisting of antiparallel homodimers. During mitosis KSP associates with microtubules of the mitotic spindle. Microinjection of antibodies directed against KSP into human cells prevents spindle pole separation during prometaphase, giving rise to monopolar spindles and causing mitotic arrest and induction of programmed cell death.

Human KSP has been described Blangy, et al., Cell, 83: 1159-69 (1995); Whitehead, et al., Arthritis Rheum., 39: 1635-42 (1996); Galgio et al., J. Cell Biol., 135: 339-414 (1996); Blangy, et al., J. Biol. Chem., 272: 19418-24 (1997); Blangy, et al., Cell Motil Cytoskeleton, 40: 174-82 (1998); Whitehead and Rattner, J. Cell Sci. 111: 2551-61 (1998); Kaiser, et al., J. Biol. Chem. 274: 18925-31 (1999); GenBank accession numbers: X85137, NM004523 and U37426. See also U.S. Pat. Nos. 6,437,115 and 6,414,121, both incorporated by reference in their entirety for all purposes. A fragment of the KSP gene (TRIPS) has also been described Lee, et al., Mol Endocrinol., 9: 243-54 (1995); and GenBank accession number L40372.

A number of KSP inhibitors have been identified. These include a large family of quinazolinone derivatives that are described in PCT publications WO 01/30768 and WO 01/98278, both of which are incorporated herein by reference in their entirety for all purposes. These inhibitors can inhibit or modulate mitotic kinesins, but not other types of kinesins (e.g., transport kinesins), thereby achieving selective inhibition of cellular proliferation. Such inhibitors are thought to function by perturbing mitotic kinesin function that results in malformation or dysfunction of mitotic spindles. This in turn frequently results in cell cycle arrest and cell death.

Because of their attractiveness as a target, further information regarding kinesins generally, and KSP in particular, would be useful in the further development of chemotherapeutic agents.

### SUMMARY

A number of nucleic acids that are differentially expressed in certain tumors or cancers are provided. These nucleic acids, or the proteins they encode, can be utilized in a variety of different methods for classifying, diagnosing and treating tumors, as well as in kits and devices for conducting such methods.

Certain classification methods, for instance, initially involve providing a test sample derived from a tumor cell, wherein the tumor cell is capable of expressing one or more nucleic acid markers selected from the group consisting of those listed in Table 1 and Table 2. The expression level of the one or more nucleic acid markers in the test sample are then determined. These expression levels are compared with the expression level of the one or more nucleic acid markers in a control sample whose tumor status is known. The tumor cell is then classified on the basis of the comparison of step.

Other methods involve determining whether a cancerous tissue is treatable with an inhibitor of KSP. Identification of such tumors can be very useful in developing a therapeutic strategy because of the attractiveness of KSP inhibitors as chemotherapeutics. These methods generally involve providing a test sample derived from a cancerous tissue from a subject. The expression levels of one or more markers from Table 1 and Table 2 in the cancerous tissue are then determined. An increase in expression of one or more markers from those listed in Table 1 and a decrease in expression of one or more markers from those listed in Table 2 relative to the levels of these markers in a normal sample of the same type of tissue is an indication that the cancerous tissue is treatable by the inhibitor of KSP.

Various diagnostics can be utilized based upon the differentially expressed genes that are identified herein. Some of these methods involve diagnosing the presence of, or predisposition to, a tumor in a subject. These methods usually involve determining the expression level of one or more nucleic acid markers in a test sample obtained from the subject, wherein the one or more nucleic acid markers are selected from the group consisting of those listed in Table 1 and Table 2. The expression level of the one or more nucleic acid markers in the test sample are then compared with the expression level of these same nucleic acid markers in a control sample whose tumor status is known. The presence or absence of the tumor in the subject, or a predisposition to the tumor by the subject, is then diagnosed on the basis of the comparison of step.

A number of different screening methods are also provided. Some of these are designed to identify an inhibitor of a tumor. Such methods generally involve contacting a test cell capable of expressing one or more nucleic acid markers selected from the group comprising those listed in Table 1 or Table 2 with a test agent. The expression level of one or more nucleic acid markers comprising those listed in Table 1 and Table 2 are then determined. The expression level of the one or more nucleic acid markers are compared with the expression level of the same markers for a control cell population whose tumor status is known and that has not been contacted with the test agent. Finally, the test agent is identified as an inhibitor of the tumor on the basis of the comparison step.

Another set of screening methods involve assessing whether a test agent is a potential carcinogen. Methods of this type typically involve contacting a test cell capable of expressing one or more nucleic acid markers selected from the group consisting of those listed in Table 1 or Table 2 with the test agent. The expression level of one or more nucleic acid markers selected from the group of those listed in Table 1 and Table 2 are then determined. These expression levels are compared with the expression level of the same markers for a control cell population that is representative of cells from tissue having the cancer and/or not having the cancer. A test agent is identified as a carcinogen on the basis of the comparison step.

Treatment methods are also provided. These are designed to counteract the up-regulation and/or down-regulation of genes that are differentially expressed in certain tumors. Some methods are designed to treat tumors having a high mitotic index. These methods involve administering to a subject having the tumor, or at risk of developing the tumor, a pharmaceutical agent that inhibits the expression or activity of one or more nucleic acid markers selected from the group consisting of those listed in Table 1 and/or activates the expression or activity of one or more nucleic acids selected from the group consisting of those listed in Table 2.

Other treatment methods are directed to treating a tumor with a low mitotic index. Methods of this type generally involve administering to a subject having the tumor, or at risk of developing the tumor, a pharmaceutical agent that activates the expression or activity of one or more nucleic acid markers selected from the group consisting of those listed in Table 1 and/or inhibits the expression or activity of one or more nucleic acids selected from the group consisting of those listed in Table 2.

### BRIEF DESCRIPTION OF THE DRAWINGS

FIG. 1 is chart showing expression of KSP in normal tissues showing increased expression in thymus, bone marrow and some expression in organs of the digestive tract such as colon, esophagus, rectum, stomach and small intestine.

FIG. 2 is a plot illustrating that expression of KSP in malignant breast tumors (breast infiltrating ductal carcinomas) as compared to normal breast tissues shows a spread in KSP expression. While KSP levels are generally increased in this group, some tumor patients have KSP levels that overlaps “normal” expression.

FIG. 3 shows the result of a Cluster Analysis. This analysis demonstrates the separation of breast tumor samples into those that show relatively higher expression of genes associated with cell cycle and those that show relatively higher expression of signal transduction. Results for each of 200 tissue samples are shown along the x-axis; results for different genes for each of the 200 individuals are shown along the y-axis. As indicated on the x-axis, tumor patients with normal KSP levels are represented at the left-hand side of the axis, whereas tumor patients with elevated KSP levels are represented at the right-hand side of the axis. The results can generally be divided into 6 regions. Regions A, B and C include genes that are primarily signal transduction genes (see Table 2). Regions D, E and F generally correspond to genes that fall within the class of cell cycle genes (see Table 1).

### DESCRIPTION

I. Definitions

A “tumor” has its normal meaning in the art and refers to an abnormal growth of tissue without physiological function. A tumor can be cancerous or benign; thus, a tumor includes a cancer.

“Mitotic index” is an indication of the number of genes expressed in a cell that are cell cycle genes, i.e., those genes that are involved in cell proliferation, specifically in mitosis. Examples of such genes include, but are not limited to, those listed in Table 1.

A “normal cell” is one that does not have the particular cancer or tumor of interest. Often such a cell is free of any type of cancer or tumor. When expression levels in a normal cell are to be compared with those in a test cell (e.g., a cell having or suspected of having a tumor), the normal cell is typically selected to be as similar as possible to the test cell, except with respect to status of the cancer or tumor of interest.

The term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof. A “subsequence” or “segment” refers to a sequence of nucleotides or amino acids that comprise a part of a longer sequence of nucleotides or amino acids (e.g., a polypeptide), respectively.

A “polynucleotide” refers to a single or double-stranded polymer of deoxyribonucleotide or ribonucleotide bases.

The term “target nucleic acid” refers to a nucleic acid (often derived from a biological sample), to which the polynucleotide probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The term target nucleic acid can refer to the specific subsequence of a larger nucleic acid to which the probe is directed or to the overall sequence (e.g., gene or mRNA) whose expression level it is desired to detect.

A “probe” or “polynucleotide probe” is an nucleic acid capable of binding to a target nucleic acid of complementary sequence through one or more types of chemical bonds, usually through complementary base pairing, usually through hydrogen bond formation, thus forming a duplex structure. The probe binds or hybridizes to a “probe binding site.” A probe can include natural (ie., A, G, C, or T) or modified bases (7-deazaguanosine, inosine, etc.). A probe can be an oligonucleotide which is a single-stranded DNA. Polynucleotide probes can be synthesized or produced from naturally occurring polynucleotides. In addition, the bases in a probe can be joined by a linkage other than a phosphodiester bond, so long as it does not interfere with hybridization. Thus, probes can include, for example, peptide nucleic acids in which the constituent bases are joined by peptide bonds rather than phosphodiester linkages (see, e.g., Nielsen et al., Science 254, 1497-1500 (1991)). Some probes can have leading and/or trailing sequences of noncomplementarity flanking a region of complementarity.

A “perfectly matched probe” has a sequence perfectly complementary to a particular target sequence. The probe is typically perfectly complementary to a portion (subsequence) of a target sequence. The term “mismatch probe” refer to probes whose sequence is deliberately selected not to be perfectly complementary to a particular target sequence.

A “primer” is a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides, although shorter or longer primers can be used as well. Short primer molecules generally require cooler temperatures to form sufficiently stable hybrid complexes with the template. A primer need not reflect the exact sequence of the template but must be sufficiently complementary to hybridize with a template. The term “primer site” refers to the area of the target DNA to which a primer hybridizes. The term “primer pair” means a set of primers including a 5′ “upstream primer” that hybridizes with the 5′ end of the DNA sequence to be amplified and a 3′ “downstream primer” that hybridizes with the complement of the 3′ end of the sequence to be amplified.

The term “complementary” means that one nucleic acid is identical to, or hybridizes selectively to, another nucleic acid molecule. Selectivity of hybridization exists when hybridization occurs that is more selective than total lack of specificity. Typically, selective hybridization will occur when there is at least about 55% identity over a stretch of at least 14-25 nucleotides, preferably at least 65%, more preferably at least 75%, and most preferably at least 90%. Preferably, one nucleic acid hybridizes specifically to the other nucleic acid. See M. Kanehisa, Nucleic Acids Res. 12:203 (1984).

The terms “polypeptide,” “peptide” and “protein” are used interchangeably to refer to a polymer of amino acid residues. The term also applies to amino acid polymers in which one or more amino acids are chemical analogues of a corresponding naturally occurring amino acids.

The term “operably linked” refers to functional linkage between a nucleic acid expression control sequence (such as a promoter, signal sequence, or array of transcription factor binding sites) and a second polynucleotide, wherein the expression control sequence affects transcription and/or translation of the second polynucleotide.

The terms “identical” or percent “identity,” in the context of two or more nucleic acids or polypeptides, refer to two or more sequences or subsequences that are the same or have a specified percentage of nucleotides or amino acid residues that are the same, when compared and aligned for maximum correspondence, as measured using a sequence comparison algorithm such as those described below for example, or by visual inspection.

The phrase “substantially identical,” in the context of two nucleic acids or polypeptides, refers to two or more sequences or subsequences that have at least 75%, preferably at least 85%, more preferably at least 90%, 95% or higher nucleotide or amino acid residue identity, when compared and aligned for maximum correspondence, as measured using a sequence comparison algorithm such as those described below for example, or by visual inspection. Preferably, the substantial identity exists over a region of the sequences that is at least about 30 residues in length, preferably over a longer region than 50 residues, more preferably at least about 70 residues, and most preferably the sequences are substantially identical over the full length of the sequences being compared, such as the coding region of a nucleotide for example. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see, e.g., Current Protocols in Molecular Biology (Ausubel et al., 1995 supplement).

One useful algorithm for conducting sequence comparisons is PILEUP. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithm that is suitable for determining percent sequence identity and sequence similarity is the BLAST and the BLAST 2.0 algorithms, which are described in Altschul et al., J. Mol. Biol. 215:403-410 (1990). Software for performing BLAST analyses is publicly available through the National Center for Biotechnology Information (http://www.ncbi.nhn.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

For identifying whether a nucleic acid or polypeptide is within the scope of the invention, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM 62 scoring matrix. The TBLATN program (using protein sequence for nucleotide sequence) uses as defaults a word length (W) of 3, an expectation (E) of 10, and a BLOSUM 62 scoring matrix. (See, e.g., Henikoff & Henikoff, Proc. Natl. Acad. Sci. USA 89:10915 (1989)).

Another indication that two nucleic acid sequences are substantially identical is that the two molecules hybridize to each other under stringent conditions. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence. The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide.

A further indication that two nucleic acid sequences or polypeptides are substantially identical is that the polypeptide encoded by the first nucleic acid is immunologically cross reactive with the polypeptide encoded by the second nucleic acid, as described below. The phrases “specifically binds to a protein” or “specifically immunoreactive with,” when referring to an antibody refers to a binding reaction which is determinative of the presence of the protein in the presence of a heterogeneous population of proteins and other biologics. Thus, under designated immunoassay conditions, a specified antibody binds preferentially to a particular protein and does not bind in a significant amount to other proteins present in the sample. Specific binding to a protein under such conditions requires an antibody that is selected for its specificity for a particular protein. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein which encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

A polypeptide is typically substantially identical to a second polypeptide, for example, where the two peptides differ only by conservative substitutions. A “conservative substitution,” when describing a protein, refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well-known in the art. See, e.g., Creighton (1984) Proteins, W.H. Freeman and Company. In addition, individual substitutions, deletions or additions which alter, add or delete a single amino acid or a small percentage of amino acids in an encoded sequence are also “conservatively modified variations.”

The term “naturally occurring” as applied to an object refers to the fact that an object can be found in nature. For example, a polypeptide or polynucleotide sequence that is present in an organism that can be isolated from a source in nature and which has not been intentionally modified by humans in the laboratory is naturally occurring.

The term “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit comprises a tetramer. Bach tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to VH-CH1 by a disulfide bond. The F(ab)′₂ may be reduced under mild conditions to break the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies, more preferably single chain Fv (scFv) antibodies in which a variable heavy and a variable light chain are joined together (directly or through a peptide linker) to form a continuous polypeptide.

A single chain Fv (“scFv” or “scFv”) polypeptide is a covalently linked VH::VL heterodimer which may be expressed from a nucleic acid including VH- and VL-encoding sequences either joined directly or joined by a peptide-encoding linker. Huston, et al. Proc. Nat. Acad. Sci. USA, 85:5879-5883 (1988). A number of structures for converting the naturally aggregated—but chemically separated light and heavy polypeptide chains from an antibody V region into an scFv molecule which will fold into a three dimensional structure substantially similar to the structure of an antigen-binding site. See, e.g. U.S. Pat. Nos. 5,091,513 and 5,132,405 and 4,956,778.

An “antigen-binding site” or “binding portion” refers to the part of an immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions” or “FRs”. Thus, the term “FR” refers to amino acid sequences that are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen binding “surface”. This surface mediates recognition and binding of the target antigen. The three hypervariable regions of each of the heavy and light chains are referred to as “complementarity determining regions” or “CDRs” and are characterized, for example by Kabat et al. Sequences of proteins of immunological interest, 4th ed. U.S. Dept. Health and Human Services, Public Health Services, Bethesda, Md. (1987).

The term “antigenic determinant” refers to the particular chemical group of a molecule that confers antigenic specificity.

The term “epitope” generally refers to that portion of an antigen that interacts with an antibody. More specifically, the term epitope includes any protein determinant capable of specific binding to an immunoglobulin or T-cell receptor. Specific binding exists when the dissociation constant for antibody binding to an antigen is ≦1 μM, preferably <100 nM and most preferably ≦1 nM. Epitopic determinants usually consist of chemically active surface groupings of molecules such as amino acids and typically have specific three dimensional structural characteristics, as well as specific charge characteristics.

The term “specific binding” (and equivalent phrases) refers to the ability of a binding moiety (e.g., a receptor, antibody, ligand or antiligand) to bind preferentially to a particular target molecule (e.g., ligand or antigen) in the presence of a heterogeneous population of proteins and other biologics (i.e., without significant binding to other components present in a test sample). Typically, specific binding between two entities, such as a ligand and a receptor, means a binding affinity of at least about 106 M-1, and preferably at least about 10⁷, 10⁸, 10⁹, or 10¹⁰ M⁻¹.

a “subject” generally refers to an organism that has a tumor. Usually the subject is a mammal (e.g., a primate such as a monkey, ape, or chimpanzee), and often is a human.

II. Overview

A variety of methods for classifying, diagnosing and treating cancers or tumors are provided, as well as kits and devices including nucleic acids, proteins and antibodies useful for performing such methods. The methods, kits and devices that are disclosed are based in part on the identification of a relatively small group of “differentially expressed nucleic acids” or “differentially expressed genes” that exhibit different expression levels between tumor cells and normal cells, or between different types of tumors. There expression level in certain tumors is also positively or negatively correlated with the kinesin motor protein KSP (see Tables 1 and 2). These differentially expressed nucleic acids and the proteins encoded by them can be utilized as “markers” for classifying and diagnosing various types of tumors.

Using a combination of techniques to analyze differential gene expression in various tumor types, it was found that certain tumors fell into three groups. In the first group, expression levels for KSP and cell cycle genes (see, e.g., Table 1) were increased such that the group was characterized by a high mitotic index, but signal transduction gene expression was decreased. The second group was characterized by elevated expression levels of signal transduction genes (see, e.g., Table 2) but normal KSP levels and decreased expression of cell cycle genes. The third group exhibited increased KSP and cell cycle gene expression, but also increased expression of signal transduction genes. This analysis also demonstrated that the cell cycle genes listed in Table 1 correlated positively with KSP expression, whereas the signal transduction genes listed in Table 2 correlated negatively with KSP expression.

Identification of the differential gene expression profiles for these different tumor types provides the basis for a variety of classification, diagnostic and treatment methods. For example, tumors can be classified into one of the foregoing three groups by determining the relative expression levels of one or more of the differentially expressed genes and assessing whether the expression level of the gene(s) is consistent with the expression levels for that gene (or genes) in the three groups. Therapeutic methods can be tailored depending upon the particular type of tumor by administering a therapeutic agent that counteracts the decrease or increase in expression level for one or more of the genes that are identified herein as being differentially expressed.

So, for instance, the classification and treatment methods can be utilized to determine if the expression levels of one or more of the differentially expressed nucleic acids is consistent with a tumor that expresses high levels of KSP (e.g., determining if the expression level of one or more markers that positively correlate with KSP are increased and/or if the expression of one or more markers that negatively correlated with KSP are decreased relative to a control). Tumors falling into this category are candidates for effective treatment with KSP inhibitors. The ability to identify such tumors using the markers identified herein is important, because, as noted earlier, treatments with KSP inhibitors offer several advantages to other chemotherapeutic methods. So one important aspect of the markers that are provided is that they can serve as surrogates for KSP.

The differentially expressed nucleic acids can also be used in screening methods to identify inhibitors of certain tumors. The general strategy is to identify candidate agents that inhibit the expression of those differentially expressed nucleic acids whose expression level is elevated in the tumor and/or activate the expression of those nucleic acids whose expression level is decreased in the tumor.

Other methods determine the expression levels of one or more of the differentially expressed nucleic acids to screen agents to ascertain if they are potential carcinogens. In these methods, a test agent is contacted with a non-cancerous cell and the expression level of one or more of the differentially expressed nucleic acids determined. An increase in the expression level of those nucleic acids that are elevated in a particular tumor and/or a decrease in expression levels of those nucleic acids that are down-regulated is an indication that the test agent is a potential carcinogen.

Kits and devices such as customized arrays for use in conducting the disclosed methods are also provided. Certain kits and devices include nucleic acid probes that can specifically hybridize to one or more of the differentially expressed nucleic acids. Other kits and devices include antibodies or other receptors that specifically bind to the proteins encoded by one or more of the differentially expressed nucleic acids. Kits and devices of this type are useful in conducting the screening and diagnostic methods that are provided.

III. Differentially Expressed Nucleic Acids and Expression Profiles

Because of the importance of KSP as a chemotherapeutic target, the current inventors conducted a series of investigations to understand the scope of KSP expression in different cell types, especially in various cancers and tumors relative to normal cells. Two general techniques were utilized to conduct these analyses: quantitative RT-PCR (specifically TAQMAN procedures) and nucleic acid microarray analyses. Both of these methods are described in greater detail infra.

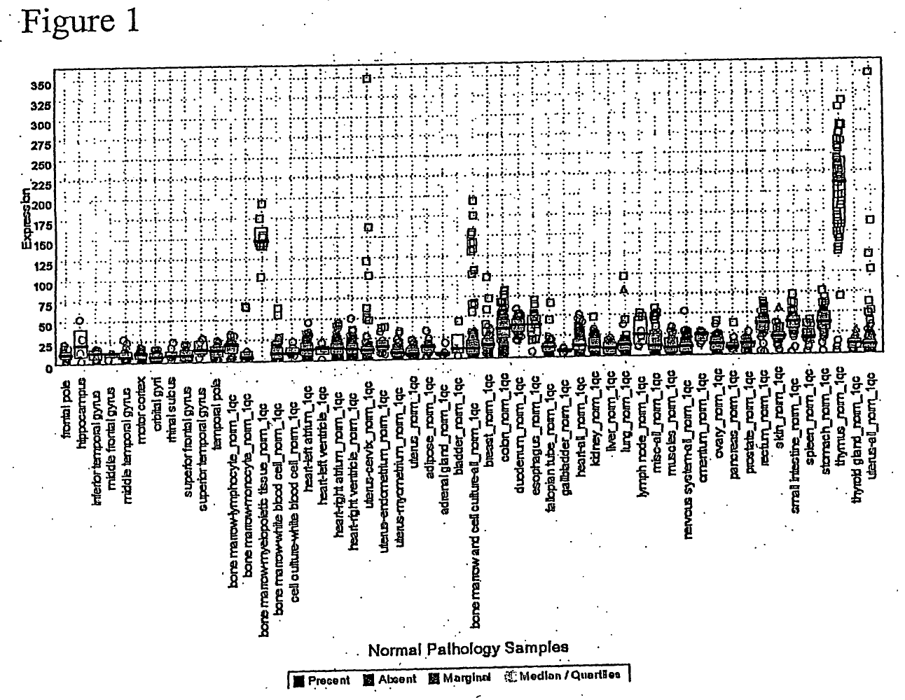

These two techniques were first utilized to investigate KSP expression levels in various types of tissues to determine if KSP is expressed ubiquitously or only in select tissues. Using a database of gene expression data, it was determined that KSP is expressed at relatively high levels only in certain cells, including bone tissue (especially marrow myelopoietic cells), thymus and, to a somewhat lesser degree, colon, esophagus, rectum, stomach and small intestine (see FIG. 1). These results thus indicated that KSP is not expressed ubiquitously. Instead, it appears to be expressed in tissues in which the cells are rapidly turned over, i.e., in tissues with high proliferative capacity. This is consistent with KSP's role in cellular proliferation.

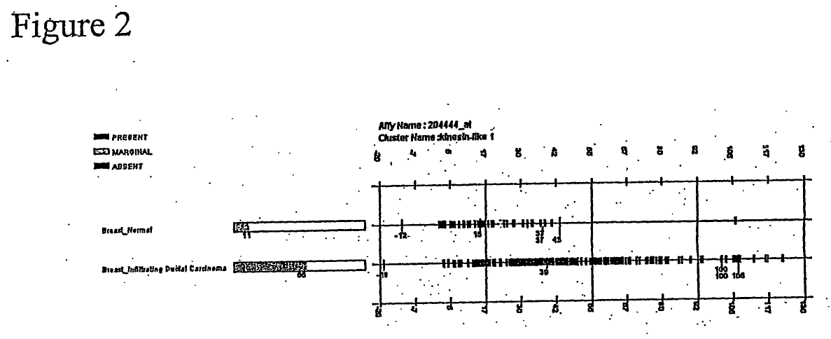

A study was then conducted to determine if KSP expression is increased in diseases involving high cellular proliferation (e.g., tumors and cancers). One set of experiments involved a determination of the level of KSP expression in normal breast tissue from 50 different individuals, as well as in 200 individuals with a breast-infiltrating ductal carcinoma (e.g., adenocarcinoma or squamous cell carcinoma). It was found from these studies that KSP expression levels were generally increased in tumor samples relative to normal samples. The results with the tumor samples, however, showed that not all tumors express high levels of KSP. Rather, KSP levels for some individuals with tumors fell in the range expected for normal tissue. So the results indicated that individuals with at least certain tumor types can be divided into two groups: one group in which KSP levels are consistent with those for normal tissue, and a second group in which KSP levels are elevated (see FIG. 2). In other malignant tissues, however, KSP expression was not increased. Prostrate tumors, for example, express undetectable levels of KSP transcript. It was also found that KSP expression is increased in certain malignant tumors (e.g., breast, ovary and lung) but not in benign tissues. Other experiments were conducted to evaluate KSP expression relative to cell-type specific genes such as Cytokeratin 18, an epithelial marker.

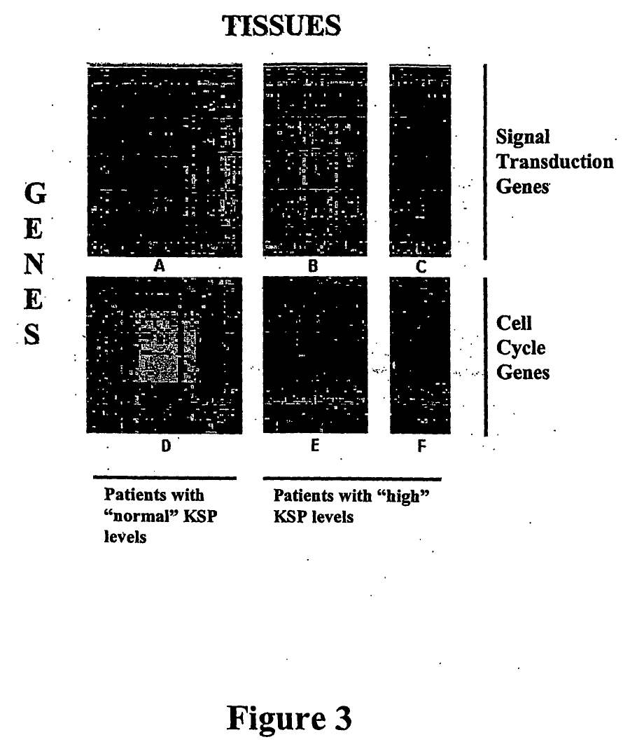

The observation that certain individuals having an infiltrating breast carcinoma have normal KSP levels whereas others have elevated levels, prompted the inventors to evaluate next whether there was a biological difference between these two groups of individuals. This was done by conducting a cluster analysis to determine if there was a difference in gene expression for samples in the two tumor groups. The genes interrogated were ones that were highly expressed in each of these two populations. As noted supra, it was discovered that the tumors could be classified into three groups: 1) those tumors characterized by increased expression of KSP (e.g., a greater than 1.5-2-fold increase in KSP expression relative to normal cells) and a high mitotic index (i.e., increased expression of cell cycle genes), but having a decreased level of signal transduction genes, 2) those tumors exhibiting increased expression of signal transduction associated genes but a decreased level of cell cycle genes, and 3) those tumors having characteristics of the other two classes, namely a high mitotic index and increased expression of signal transduction associated genes (see FIG. 3). For ease of reference, these classes of tumors will sometimes simply be referred to herein as Category 1, 2 and 3 tumors, respectively.

So one result of this investigation was the identification of a panel of nucleic acids that are positively or negatively correlated with KSP expression. Nucleic acids that correlate positively are ones whose expression tracks that of KSP (i.e., expression is increased if KSP expression is increased and decreased if KSP expression is decreased). Nucleic acids that are negatively correlated are those whose expression levels move opposite to KSP levels (i.e., the level of gene expression decreases if KSP expression levels are elevated or is increased if KSP expression levels are decreased with respect to normal cells). These nucleic acids can thus serve as markers for KSP expression.

Differentially expressed nucleic acids that positively correlate with KSP expression levels in breast tumors are shown in Table 1. These genes tend to be “cell cycle” genes, namely genes that are involved in cellular proliferation, particularly mitosis (e.g., Ki67 and Cyclin B1). Those genes that negatively correlate with KSP expression levels are shown in Table 2. Many of these genes are signal transduction genes, but genes involved in various other cellular processes are also included (see, e.g., the various functions listed in Table 4). Working from left to right on Table 1, the first column is a number for each differentially expressed gene (i.e., Differential Gene No.); the second column is a Clone ID No., which is an internal reference number assigned to each differentially expressed nucleic acid that was identified; the third column is the GenBank Accession No.; the fourth column lists the Locus Link ID; the final column provides the name of the gene commonly used in the scientific literature. Table 2 includes an additional column labeled “Alias,” which provides another common name for the gene. Collectively, the genes listed in Tables 1 and 2 are the differentially expressed nucleic acids or genes of the invention.

Studies similar to those performed with the breast infiltrating ductal carcinoma samples were also performed with samples from tumors of the ovary and lung. Based on gene expression profiles, it was found that these tumors also fell into the same three categories. To identify those genes showing the highest correlation, an additional analysis was conducted to identify those genes that were consistently up- or down-regulated in the breast, ovary and lung tumors. Those genes found to have the highest positive and negative correlation with KSP expression in these three sets of tumors are listed in Tables 3 and 4, respectively. These tables are organized as described for tables 1 and 2.

As discussed in greater detail below, knowledge of the nucleic acids that are up-regulated or down-regulated in the various tumor types provides the basis for a number of different screening, treatment and diagnostic methods, in addition to devices to carry out these methods. For instance, the differentially expressed nucleic acids include both “fingerprint genes” and “target genes.” Fingerprint genes” are those nucleic acids that correlate with a particular tumor type, or a particular cellular state (e.g., malignant or benign). As described in greater detail below, fingerprint genes can be used in the development of a variety of different screening and diagnostic methods to classify tumors and/or identify the presence or absence of a particular disease state. A “target gene” is a nucleic acid encoding a protein that causes or inhibits the formation of a tumor. If the target gene encodes a protein that is a causative agent, then down-regulation of the target gene product has a protective function. On the other hand, if a target gene encodes an inhibitory protein, then up-regulation of the target gene has a protective function. Because of their role in cancer or tumor, formation; target genes are useful targets for the development of compound discovery programs and pharmaceutical development such as described infra. In some instances, a fingerprint gene can be a target gene and vice versa.

Expression levels for combinations of differentially expressed genes, in particular fingerprint genes, can be used to develop “expression profiles” that are characteristic of a particular cancer, tumor or cellular state. Expression profiles as used herein refers to the pattern of gene expression corresponding to at least two differentially expressed genes. Typically, an expression profile includes at least 1, 2, 3, 4 or 5 differentially expressed genes, but in other instances can include at least 6, 7, 8, 9, 10, 12, 14, 16, 18, 20, 25, 30, 35, 40, 45, 50, 60, 70, 80, 90, 100 or more differentially expressed genes. In some instances, expression profiles include all of the differentially expressed genes known for a particular tumor, cancer or cellular state. So, for example, certain expression profiles include a measure (quantitative or qualitative) of the expression level for each of the differentially expressed genes in Tables 1 and 2, or Tables 3 and 4.

The pattern of expression associated with gene expression profiles can be defined in several ways. For example, a gene expression profile can be the absolute (e.g., measured value) or relative transcript level of any number of particular differentially expressed genes. In other instances, a gene expression profile can be defined by comparing the level of expression of a variety of genes in one state to the level of expression of the same genes in another state (e.g., malignant versus benign), or between one cell type and another cell type (e.g., cancerous cells versus normal cells).

As used herein, the term “differentially expressed nucleic acid” refers to the specific sequence as set forth in the particular GenBank and Locus Link ID entry as indicated in Tables 1-4. The term, however, is also intended to include more broadly naturally occurring sequences (including allelic variants of those listed for the GenBank entries), as well as synthetic and intentionally manipulated sequences (e.g., nucleic acids subjected to site-directed mutagenesis). Differentially expressed nucleic acids also include sequences that are complementary to the listed sequences, as well as degenerate sequences resulting from the degeneracy of the genetic code. Thus, the differentially expressed nucleic acids include: (a) nucleic acids having sequences corresponding to the sequences as provided in the listed GenBank accession number; (b) nucleic acids that encode amino acids encoded by the nucleic acids of (a); (c) a nucleic acid that hybridizes under stringent conditions to a complement of the nucleic acid of (a); and (d) nucleic acids that hybridize under stringent conditions to, and therefore are complements of, the nucleic acids described in (a) through (c). The differentially expressed nucleic acids of the invention also include: (a) a deoxyribonucleotide sequence complementary to the full-length nucleotide sequences corresponding to the listed GenBank accession numbers; (b) a ribonucleotide sequence complementary to the full-length sequence corresponding to the listed GenBank accession numbers; and (c) a nucleotide sequence complementary to the deoxyribonucleotide sequence of (a) and the ribonucleotide sequence of (b). The differentially expressed nucleic acids further include fragments of the foregoing sequences. For example, nucleic acids including 10, 12, 14, 16, 18, 20, 22, 24, 26, 28, 30, 40, 50, 60, 70, 80, 90, 100, 125, 150, 175, 200, 225, 250, 275 or 300 contiguous nucleotides (or any number of nucleotides therebetween) from a differentially expressed nucleic acid are included. Such fragments are useful, for example, as primers and probes for hybridizing full-length differentially expressed nucleic acids (e.g., in detecting and amplifying such sequences).

In some instances, the differentially expressed nucleic acids include conservatively modified variations. Thus, for example, in some instances, the differentially expressed nucleic acids are modified. One of skill will recognize many ways of generating alterations in a given nucleic acid construct. Such well-known methods include site-directed mutagenesis, PCR amplification using degenerate polynucleotides, exposure of cells containing the nucleic acid to mutagenic agents or radiation and chemical synthesis of a desired polynucleotide (e.g., in conjunction with ligation and/or cloning to generate large nucleic acids). See, e.g., Giliman and Smith (1979) Gene 8:81-97, Roberts et al. (1987) Nature 328: 731-734). When the differentially expressed nucleic acids are incorporated into vectors, the nucleic acids can be combined with other sequences including, but not limited to, promoters, polyadenylation signals, restriction enzyme sites and multiple cloning sites. Thus, the overall length of the nucleic acid can vary considerably.

Certain differentially expressed nucleic acids of the invention include polynucleotides that are substantially identical to a polynucleotide sequence as set forth in SEQ ID NO:1. Such nucleic acids can function as new markers for certain types of tumors. For example, the invention includes polynucleotide sequences that are at least 80%, 85%, 90%, 92%, 94%, 96%, 98% or 100% identical to the polynucleotide sequences provided in the GenBank entries listed in Tables 1-4. Identity is typically measured over at least 40, 50, 60, 70, 80, 90 or 100 contiguous nucleotides. In other instances, identity is measured over a region of at least 150, 200, or 250 nucleotides in length. In yet other instances, the region of similarity exceeds 250 nucleotides in length and extends for at least 300, 350, 400, 450 or 500 nucleotides in length, or over the entire length of the sequence.

As described above, sequence identity comparisons can be conducted using a nucleotide sequence comparison algorithm such as those know to those of skill in the art. For example, one can use the BLASTN algorithm. Suitable parameters for use in BLASTN are wordlength (W) of 11, M=5 and N=−4 and the identity values and region sizes just described.

B. Preparation of Differentially Expressed Genes

The differentially expressed nucleic acids can be obtained by any suitable method known in the art, including, for example: (1) hybridization of genomic or cDNA libraries with probes to detect homologous nucleotide sequences; (2) antibody screening of expression libraries to detect cloned DNA fragments with shared structural features; (3) various amplification procedures such as polymerase chain reaction (PCR) using primers capable of annealing to the nucleic acid of interest; and (4) direct chemical synthesis.

The desired nucleic acids can also be cloned using well-known amplification techniques. Examples of protocols sufficient to direct persons of skill through in vitro amplification methods, including the polymerase chain reaction (PCR) the ligase chain reaction (LCR), Qβ-replicase amplification and other RNA polymerase mediated techniques, are found in Berger, Sambrook, and Ausubel, as well as Mullis et al. (1987) U.S. Pat. No. 4,683,202; PCR Protocols A Guide to Methods and Applications (Inis et al. eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039.

As an alternative to cloning a nucleic acid, a suitable nucleic acid can be chemically synthesized. Direct chemical synthesis methods include, for example, the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method described in U.S. Pat. No. 4,458,066. Chemical synthesis produces a single stranded polynucleotide. This can be converted into double stranded DNA by hybridization with a complementary sequence, or by polymerization with a DNA polymerase using the single strand as a template. While chemical synthesis of DNA is often limited to sequences of about 100 bases, longer sequences can be obtained by the ligation of shorter sequences. Alternatively, subsequences can be cloned and the appropriate subsequences cleaved using appropriate restriction enzymes. The fragments can then be ligated to produce the desired DNA sequence.

C. Utility of Differentially Expressed Nucleic Acids and Expression Profiles

As alluded to above and described in greater detail below, the differentially expressed nucleic acids and expression profiles that are provided can be used as markers in a variety of screening and diagnostic methods. For example, the differentially expressed nucleic acids find utility as hybridization probes or amplification primers. In certain instances, these probes and primers are fragments of the differentially expressed nucleic acids of the lengths described earlier in this section. Such fragments are generally of sufficient length to specifically hybridize to an RNA or DNA in a sample obtained from a subject. The nucleic acids are typically 10-30 nucleotides in length, although they can be longer as described above. The probes can be used in a variety of different types of hybridization experiments, including, but not limited to, Northern blots and Southern blots and in the preparation of custom arrays (see infra). The differentially expressed nucleic acids can also be used in the design of primers for amplifying the differentially expressed nucleic acids and in the design of primers and probes for quantitative RT-PCR. The primers most frequently include about 20 to 30 contiguous nucleotides of the differentially expressed nucleic acids to obtain the desired level of stability and thus selectivity in amplification, although longer sequences as described above can also be utilized.

Hybridization conditions are varied according to the particular application. For applications requiring high selectivity (e.g., amplification of a particular sequence), relatively stringent conditions are utilized, such as 0.02 M to about 0.10 M NaCl at temperatures of about 50° C. to about 70° C. High stringency conditions such as these tolerate little, if any, mismatch between the probe and the template or target strand of the differentially expressed nucleic acid. Such conditions are useful for isolating specific genes or detecting particular mRNA transcripts, for example.

Other applications, such as substitution of amino acids by site-directed mutagenesis, require less stringency. Under these conditions, hybridization can occur even though the sequences of the probe and target nucleic acid are not perfectly complementary, but instead include one or more mismatches. Conditions can be rendered less stringent by increasing the salt concentration and decreasing temperature. For example, a medium stringency condition includes about 0.1 to 0.25 M NaCl at temperatures of about 37° C. to about 55° C. Low stringency conditions include about 0.1 5M to about 0.9 M salt, at temperatures ranging from about 20° C. to about 55° C.

V. Proteins

A. General

The differentially expressed nucleic acids that have been identified can be inserted into any of a number of known expression systems to generate large amounts of the protein encoded by the gene or gene fragment. Such proteins can then be utilized in the preparation of antibodies. Proteins encoded by target genes can be utilized in the compound development programs described below and in the preparation of various diagnostics (e.g., antibody arrays).

The polypeptides can be isolated from natural sources, and/or prepared according to recombinant methods, and/or prepared by chemical synthesis, and/or prepared using a combination of recombinant methods and chemical synthesis. Besides substantially full-length polypeptides, biologically active fragments of the polypeptides are also provided. Biological activity can include, for example, antibody binding (e.g. the fragment competes with a full-length polypeptide) and immunogenicity (i.e., possession of epitopes that stimulate B- or T-cell responses against the fragment). Such fragments generally comprise at least 5 contiguous amino acids, typically at least 6 or 7 contiguous amino acids, in other instances 8 or 9 contiguous amino acids, usually at least 10, 11 or 12 contiguous amino acids, in still other instances at least 13 or 14 contiguous amino acids, in yet other instances at least 16 contiguous amino acids, and in some cases at least 20, 40, 60 or 80 contiguous amino acids.

Often the polypeptides will share at least one antigenic determinant in common with the amino acid sequence of the full-length polypeptide. The existence of such a common determinant is evidenced by cross-reactivity of the variant protein with any antibody prepared against the full-length polypeptide. Cross-reactivity can be tested using polyclonal sera against the full-length polypeptide, but can also be tested using one or more monoclonal antibodies against the full-length polypeptide.

The polypeptides include conservative variations of the naturally occurring polypeptides. Such variations can be minor sequence variations of the polypeptide that arise due to natural variation within the population (e.g., single nucleotide polymorphisms) or they can be homologs found in other species. They also can be sequences that do not occur naturally but that are sufficiently similar so that they function similarly and/or elicit an immune response that cross-reacts with natural forms of the polypeptide. Sequence variants can be prepared by standard site-directed mutagenesis techniques. The polypeptide variants can be substitutional, insertional or deletion variants. Deletion variants lack one or more residues of the native protein that are not essential for function or immunogenic activity (e.g., polypeptides lacking transmembrane or secretory signal sequences). Substitutional variants involve conservative substitutions of one amino acid residue for another at one or more sites within the protein and can be designed to modulate one or more properties of the polypeptide such as stability against proteolytic cleavage. Insertional variants include, for example, fusion proteins such as those used to allow rapid purification of the polypeptide and also can include hybrid proteins containing sequences from other polypeptides which are homologues of the polypeptide. The foregoing variations can be utilized to create equivalent, or even an improved, second-generation polypeptide. Preparation of variants is well known in the art (see, e.g., Creighton (1984) Proteins, W.H. Freeman and Company, which is incorporated herein by reference in its entirety for all purposes).

The polypeptides that are provided also include those in which the polypeptide has a modified polypeptide backbone. Examples of such modifications include chemical derivatizations of polypeptides, such as acetylations and carboxylations. Modifications also include glycosylation modifications and processing variants of a typical polypeptide. Such processing steps specifically include enzymatic modifications, such as ubiquitinization and phosphorylation. See, e.g., Hershko & Ciechanover, Ann. Rev. Biochem. 51:335-364 (1982). Also included are mimetics which are peptide-containing molecules that mimic elements of protein secondary structure (see, e.g., Johnson, et al., “Peptide Turn Mimetics” in Biotechnology and Pharmacy, (Pezzuto et al., Eds.), Chapman and Hall, New York (1993)). Peptide mimetics are typically designed so that side chain groups extending from the backbone are oriented such that the side chains of the mimetic can be involved in molecular interactions similar to the interactions of the side chains in the native protein.

B. Production of Polypeptides

1. Recombinant Technologies

The polypeptides encoded by the differentially expressed nucleic acids can be expressed in hosts after the coding sequences have been operably linked to an expression control sequence in an expression vector. Expression vectors are typically replicable in the host organisms either as episomes or as an integral part of the host chromosomal DNA. Expression vectors commonly contain selection markers, e.g., tetracycline resistance or hygromycin resistance, to permit detection and/or selection of those cells transformed with the desired DNA sequences (see, e.g., U.S. Pat. No. 4,704,362).

A differentially expressed gene typically is placed under the control of a promoter that is functional in the desired host cell to produce relatively large quantities of a polypeptide of the invention. An extremely wide variety of promoters are well known to those of skill, and can be used in the expression vectors, depending on the particular application. Ordinarily, the promoter selected depends upon the cell in which the promoter is to be active. Other expression control sequences such as ribosome binding sites, transcription termination sites and the like are also optionally included. Constructs that include one or more of such control sequences are termed “expression cassettes.” Accordingly, expression cassettes are provided into which the differentially expressed nucleic acids are incorporated for high level expression of the corresponding protein in a desired host cell.

In certain instances, the expression cassettes are useful for expression of polypeptides in prokaryotic host cells. Commonly used prokaryotic control sequences (defined herein to include promoters for transcription initiation, optionally with an operator, along with ribosome binding site sequences) include such commonly used promoters as the beta-lactamase (penicillinase) and lactose (lac) promoter systems (Change et al. (1977) Nature 198: 1056), the tryptophan (trp) promoter system (Goeddel et al. (1980) Nucleic Acids Res. 8: 4057), the tac promoter (DeBoer et al. (1983) Proc. Natl. Acad. Sci. U.S.A. 80:21-25); and the lambda-derived P_{L} promoter and N-gene ribosome binding site (Shimatake et al. (1981) Nature 292: 128). In general, however, any available promoter that functions in prokaryotes can be used.

For expression of polypeptides in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli.

For expression of the polypeptides in yeast, convenient promoters include GAL1-10 (Johnson and Davies (1984) Mol. Cell. Biol. 4:1440-1448) ADH2 (Russell et al. (1983) J. Biol. Chem. 258:2674-2682), PHO5 (EMBO J. (1982) 6:675-680), and MFα (Herskowitz and Oshima (1982) in The Molecular Biology of the Yeast Saccharomyces (eds. Strathern, Jones, and Broach) Cold Spring Harbor Lab., Cold Spring Harbor, N.Y., pp. 181-209). Another suitable promoter for use in yeast is the ADH2/GAPDH hybrid promoter as described in Cousens et al., Gene 61:265-275 (1987). Other promoters suitable for use in eukaryotic host cells are well-known to those of skill in the art.

For expression of the polypeptides in mammalian cells, convenient promoters include CMV promoter (Miller, et al., BioTechniques 7:980), SV40 promoter (de la Luma, et al., (1998) Gene 62:121), RSV promoter (Yates, et al, (1985) Nature 313:812), MMTV promoter (Lee, et al., (1981) Nature 294:228).

For expression of the polypeptides in insect cells, the convenient promoter is from the baculovirus Autographa Californica nuclear polyhedrosis virus (NcMNPV) (Kitts, et al., (1993) Nucleic Acids Research 18:5667).

Either constitutive or regulated promoters can be used in the expression systems. Regulated promoters can be advantageous because the host cells can be grown to high densities before expression of the polypeptides is induced. High level expression of heterologous proteins slows cell growth in some situations. For E. coli and other bacterial host cells, inducible promoters include, for example, the lac promoter, the bacteriophage lambda P_{L} promoter, the hybrid trp-lac promoter (Amann et al. (1983) Gene 25: 167; de Boer et al. (1983) Proc. Nat'l. Acad. Sci. USA 80: 21), and the bacteriophage T7 promoter (Studier et al. (1986) J. Mol. Biol.; Tabor et al. (1985) Proc. Nat'l. Acad. Sci. USA 82: 1074-8). These promoters and their use are discussed in Sambrook et al., Molecular Cloning: A Laboratory Manual, Cold Spring Harbor Press, N.Y., (1989). Inducible promoters for other organisms are also well known to those of skill in the art. These include, for example, the arabinose promoter, the lacZ promoter, the metallothionein promoter, and the heat shock promoter, as well as many others.

Construction of suitable vectors containing one or more of the above listed components employs standard ligation. Isolated plasmids or DNA fragments are cleaved, tailored, and re-ligated in the form desired to generate the plasmids required. To confirm correct sequences in plasmids constructed, the plasmids can be analyzed by standard techniques such as by restriction endonuclease digestion, and/or sequencing according to known methods. A wide variety of cloning and in vitro amplification methods suitable for the construction of recombinant nucleic acids is described, for example, in Berger and Kimmel, Guide to Molecular Cloning Techniques, Methods in Enzymology, Volume 152, Academic Press, Inc., San Diego, Calif. (Berger); and “Current Protocols in Molecular Biology,” F. M. Ausubel et al., eds., Current Protocols, a joint venture between Greene Publishing Associates, Inc. and John Wiley & Sons, Inc., (1998 Supplement) (Ausubel).

There are a variety of suitable vectors suitable for use as starting materials for constructing the expression vectors containing the differentially expressed nucleic acids of the invention. For cloning in bacteria, common vectors include pBR322-derived vectors such as PBLUESCRIPT™, pUC18/19, and λ-phage derived vectors. In yeast, suitable vectors include Yeast Integrating plasmids (e.g., YIp5) and Yeast Replicating plasmids (the YRp series plasmids) pYES series and pGPD-2 for example. Expression in mammalian cells can be achieved, for example, using a variety of commonly available plasmids, including pSV2, pBC12BI, and p91023, pcDNA series, pCMV1, pMAMneo, as well as lytic virus vectors (e.g., vaccinia virus, adenovirus), episomal virus vectors (e.g., bovine papillomavirus), and retroviral vectors (e.g., murine retroviruses). Expression in insect cells can be achieved using a variety of baculovirus vectors, including pFastBac1, pFastBacHT series, pBluesBac4.5, pBluesBacHis series, pMelBac series, and pVL1392/1393, for example.

The polypeptides encoded by the full-length genes or fragments thereof can be expressed in a variety of host cells, including E. coli, other bacterial hosts, yeast, and various higher eukaryotic cells such as the COS, CHO, HeLa and myeloma cell lines. The host cells can be mammalian cells, plant cells, insect cells or microorganisms, such as, for example, yeast cells, bacterial cells, or fungal cells. Examples of useful bacteria include, but are not limited to, Escherichia, Enterobacter, Azotobacter, Erwinia, Klebsielia.

The expression vectors can be transferred into the chosen host cell by well known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant polypeptides can be purified according to standard procedures of the art, including ammonium sulfate precipitation, affinity columns, ion exchange and/or size exclusivity chromatography, gel electrophoresis and the like (see, generally, R. Scopes, Protein Purification, Springer-Verlag, N.Y. (1982), Deutscher, Methods in Enzymology Vol. 182: Guide to Protein Purification, Academic Press, Inc. N.Y. (1990)). The polypeptides are usually purified to obtain substantially pure compositions of at least about 90 to 95% homogeneity; in other applications, the polypeptides are further purified to at least 98 to 99% or more homogeneity.

2. Naturally Occurring Polypeptides

Naturally occurring polypeptides encoded by the differentially expressed nucleic acids can also be isolated using conventional techniques such as affinity chromatography. For example, polyclonal or monoclonal antibodies can be raised against the polypeptide of interest and attached to a suitable affinity column by well-known techniques. See, e.g., Hudson & Hay, Practical Immunology (Blackwell Scientific Publications, Oxford, UK, 1980), Chapter 8 (incorporated by reference in its entirety). Peptide fragments can be generated from intact polypeptides by chemical or enzymatic cleavage methods known to those of skill in the art.

3. Other Methods

Alternatively, the polypeptides encoded by differentially expressed genes or gene fragments can be synthesized by chemical methods or produced by in vitro translation systems using a polynucleotide template to direct translation. Methods for chemical synthesis of polypeptides and in vitro translation are well-known in the art, and are described further by Berger & Kimmel, Methods in Enzymology, Volume 152, Guide to Molecular Cloning Techniques, Academic Press, Inc., San Diego, Calif., 1987 (incorporated by reference in its entirety).

C. Utility

The polypeptides can be used to generate antibodies that specifically bind to epitopes associated with the polypeptides or fragments thereof. Commercially available computer sequence analysis can be used to determine the location of the predicted major antigenic determinant epitopes of the polypeptide (e.g., MacVector from IBI, New Haven, Conn.). Once such an analysis has been performed, polypeptides can be prepared that contain at least the essential structural features of the antigenic determinant and can be utilized in the production of antisera against the polypeptide. Minigenes or gene fusions encoding these determinants can be constructed and inserted into expression vectors such as those described above using standard techniques. The major antigenic determinants can also be determined empirically in which portions of the gene encoding the polypeptide are expressed in a recombinant host, and the resulting proteins tested for their ability to elicit an immune response. For example, PCR can be used to prepare a range of cDNAs encoding polypeptides lacking successively longer fragments of the C-terminus of the polypeptide. The immunoprotective activity of each of these polypeptides then identifies those fragments or domains of the polypeptide that are essential for this activity. Further experiments in which only a small number or amino acids are removed at each iteration then allows the location of the antigenic determinants of the polypeptide.

Polypeptides encoded by target genes can be utilized in the development of pharmaceutical compositions, for example, that modulate gene products associated cancerous cells. The process for identifying such polypeptides and subsequent compound development is described further below.

VI. Exemplary Screening, Classification and Diagnostic Methods

A. General Considerations

A number of the methods that are provided involve determining the expression level of one or more of the differentially expressed nucleic acids in a test cell population with the expression level of the same nucleic acids in a control cell population. The level of expression of the differentially expressed nucleic acids can be determined at either the nucleic acid level or the protein level. Thus, the phrase “determining the expression level” and other like phrases when used in reference to the differentially expressed nucleic acids means that transcript levels and/or levels of protein encoded by the differentially encoded nucleic acids are detected. When determining the level of expression, the level can be determined qualitatively, but generally is determined quantitatively.

Based upon the sequence information that is disclosed herein, coupled with the nucleic acid and protein detection methods that are described herein and that are known in the art, expression levels of these genes can readily determined. If transcript levels are determined, they can be determined using routine methods. For instance, the sequence information provided herein (e.g., GenBank sequence entries) can be used to construct nucleic acid probes using conventional methods such as various hybridization detection methods (e.g., Northern blots). Alternatively, the provided sequence information can be used to generate primers that in turn are used to amplify and detect differentially expressed nucleic acids that are present in a sample (e.g., quantitative RT-PCR methods). If instead expression is detected at the protein level, encoded protein can be detected and optionally quantified using any of a number of established techniques. One common approach is to use antibodies that specifically bind to the protein product in immunoassay methods. Additional details regarding methods of conducting differential gene expression are provided infra.

Expression levels can be detected for one, some, or all of the differentially expressed nucleic acids that are listed in Tables 1-4. With some methods, the expression levels for only 1, 2, 3, 4 or 5 differentially expressed nucleic acids are determined. In other methods, expression levels for at least 6, 7, 8, 9 or 10 differentially expressed nucleic acids are determined. In still other methods, expression levels for at least 15, 20, 25, 30, 35, 40, 45, 50, 55, or 60 differentially expressed nucleic acids are determined. In yet other methods, all of the differentially expressed genes in Tables 1 and 2 are determined, or alternatively all those listed in Tables 3 and 4 are determined. Some methods also involve the determination of expression levels for KSP and/or tubulin.

Determination of expression levels is typically done with a test sample taken from a test cell population. As used herein, the term “population” when used in reference to a cell can mean a single cell, but typically refers to a plurality of cells (e.g., a tissue sample). The test cell population can include a plurality of different cell types, but typically includes a single cell type. In certain methods (e.g., classification or diagnostic methods), the test sample is usually obtained from a tumor or cancerous tissue, or from a tissue thought to contain a tumor or be cancerous.

Certain screening methods (e.g., screening to assess whether a test agent is a carcinogen) typically use test cells that are not from a tumor and are not cancerous. Methods of this type are performed with test cells that are “capable of expressing” one or more of the differentially expressed nucleic acids. As used in this context, the phrase “capable or expressing” means that the nucleic acid of interest is in intact form and can be expressed within the cell.

Essentially any type of cell can be used in the screening methods that are provided so long as it is capable of expressing one or more of the differentially expressed nucleic acids. Examples of such cells or those obtained from a variety of different human tissues including, but not limited to, liver, breast, skin, kidney, stomach and pancreas. Suitable cells lines include, for example, HepG2, HeLa, HL60 and MCF7 cells.

A number of the methods that are provided involve a comparison of expression levels for certain differentially expressed nucleic acids in a “test cell” with the expression levels for the same nucleic acids in a “control cell” (also sometimes referred to as a “control sample,” a “reference cell,” a “reference value,” or simply a “control”). The expression level for the control cell essentially establishes a baseline against which an experimental value is compared. The comparison of expression levels are meant to be interpreted broadly with respect to what is meant by: 1) the term “cell”, 2) the time at which the expression levels for test and control cells are determined, and 3) with respect to the measure of the expression levels.

So, for example, although the term “test cell” and “control cell” is used for convenience, the term “cell” is meant to be construed broadly. A cell, for instance, can also refer to a population of cells (e.g., a tissue sample), just as a population of cells can have a single member. The cell may in some instances be a sample that is derived from a cell (e.g., a cell lysate, a homogenate, a cell fraction or a cell organelle). Samples obtained from human subjects can be obtained from essentially any source from which the differentially expressed nucleic acids or their protein products can be obtained. If the method seeks to determine whether a sample is from a tumor or cancerous tissue, than the sample should be obtained from the suspicious tissue. In general, however, samples can be obtained, for example, from sputum, tissue, blood, tissue or fine-needle biopsy samples, urine, peritoneal fluid, and fleural fluid, or cells there from. Biological samples can also include sections of tissues such as frozen sections taken for histological purposes

If the control cell is an actual cell, the test and control cells generally are derived from tissues that are as similar to one another as possible. In some instances, this means that the control cell is obtained from the same subject as the test cell. So in some methods, the control cell is taken from a site proximal to the region from which the test cell is taken. For example, a control cell may be taken from normal tissue that is adjacent to tumor tissue or tissue suspected to be cancerous. Alternatively, a cell population is divided into a test and control subpopulation. The subpopulations are obtained by dividing the original sample into groups that are as nearly identical as possible. This may be the case, for instance, in in vitro or ex vivo screening methods.

With respect to timing, comparison of expression levels can be done contemporaneously (e.g., a test and control cell are each contacted with a test agent in parallel reactions). The comparison alternatively can be conducted with expression levels that have been determined at temporally distinct times. As an example, expression levels for the control cell can be collected prior to the expression levels for the test cell and stored for future use (e.g., expression levels stored on a computer compatible storage medium).

The expression level for a control cell (e.g., baseline) can be a value for a single cell or it can be an average, mean or other statistical value determined for a plurality of cells. As an example, the expression level for a control cell can be the average of the expression levels for a population of subjects (e.g., non-diseased subjects). In other instances, the value for each expression level for the control cell is a range of values representative of the range observed for a particular population. Expression level values can also be either qualitative or quantitative. The values for expression levels can also optionally be normalized with respect to the expression level of a nucleic acid that is not one of the markers under analysis.

The comparative analysis required in some methods involves determining whether the expression level values are “comparable” (or similar”), or “differ” from one another. In some instances, the expression levels for a particular marker in test and control cells are considered similar if they differ from one another by no more than the level of experimental error. Often, however, expression levels are considered similar if the level in the test cell differs by less than 5%, 10%, 20%, 50%, 100%, 150%, or 200% with respect to the control cell. It thus follows that in some instances the expression level for a particular marker in the test cell is considered to differ from the expression level for the same marker in the control cell if the difference is greater than the level of experimental error, or if it is greater than 5%, 10%, 20%, 50%, 100%, 150% or 200%. In some methods, the comparison involves a determination of whether there is a “statistically significant difference” in the expression level for a marker in the test and control cells. A difference is generally considered to be “statistically significant” if the probability of the observed difference occurring by chance (the p-value) is less than some predetermined level. As used herein a “statistically significant difference” refers to a p-value that is <0.05, preferably <0.01 and most preferably <0.001. If gene expression is increased sufficiently such that it is different (as just defined) relative to the control cell or baseline, the expression of that gene is considered “up-regulated” or “increased.” If, instead, gene expression is decreased so it differs from the control cell or baseline, the expression of that gene is “down-regulated” or “decreased.”

Comparison of the expression levels between test and control cells can involve comparing levels for a single marker or a plurality of markers as indicated above. When the expression level for a single marker is determined, whether expression levels between the test and control cell are similar or different involves a comparison of the expression level of the single marker. When, however, expression levels for multiple markers are compared, the comparison analysis often involves two analyses: 1) a determination for each marker examined whether the expression level is similar between the test and control cells, and 2) a determination of how many markers from the group of markers examined show similar or different expression levels. The first determination is done as just described. The second determination typically involves determining whether at least 50% of the markers examined show similarity in expression levels. However, in methods were more stringent correlations are required, at least 60%, 70%, 80%, 90%, 95% or 100% of the markers must show similar expression levels for the expression levels of the group of markers examined considered to be similar between the test and control cells.

B. Classifying Tumors

The current differentially expressed nucleic acids or markers either correlate positively (Tables 1 and 3) or negatively (Tables 2 and 4) with KSP expression levels. Because KSP expression is increased in certain tumor types but not others, the markers listed in these tables can be used as surrogates for KSP (or alternatively in combination with KSP) to classify tumors into different general classes or types. As an example, the results provided herein indicate that the identified markers can be utilized to classify tumors into three different categories. Classification of tumors in this way is important because different tumor types are potentially responsive to different treatment regimes. So classification can provide medical professionals with guidance on appropriate treatment options.

These classification methods generally involve obtaining a sample from a tumor cell (e.g., cancer cell) from a subject. The expression levels for one or more of the differentially expressed nucleic acids is then determined. These expression levels are subsequently compared to the level of expression in a control cell (baseline) whose tumor status is known (i.e., present or absent). Similarity or difference in expression levels with respect to the control can be used to classify the test sample as belonging to a particular class of tumor or excluded from a class. So, for example, in some methods expression levels are compared against a control cell or baseline that is representative of a known cancer or tumor. Similarity in expression levels or expression profiles between the test and control cells is an indication that the test cell is from a tumor or cancer that is within the same class or type as the control. A difference in expression levels or profiles, however, is an indication that the test cell is from a different type of tumor or cancer than the control.

One specific example of the utility of this general method involves determining whether a tumor or cancerous tissue is likely to be responsive to treatment with KSP inhibitors. As noted previously, KSP inhibitors are attractive chemotherapeutics because they are less susceptible to unwanted side effects. Because the markers that are identified herein correlate positively or negatively with KSP, they can be used to determine whether a particular tumor or cancerous tissue is one that expresses high levels of KSP, and thus whether it is a good candidate for treatment with KSP inhibitors. The method is similar to the classification methods. Expression levels for one or more of the differentially expressed nucleic acids are determined for tissue taken from a tumor or cancerous tissue. These expression levels are then compared with the expression levels for the same nucleic acids for tumor or cancerous tissue in which KSP levels are increased and/or compared against expression levels from normal tissue. As indicated above, “normal tissue” is tissue that usually is from the same type of tissue as that from which the test sample is taken. It also is typically from tissue free from tumors (e.g., non-cancerous tissue). If the comparison is made with respect to expression levels in cancerous tissue in which KSP expression is increased, then similarity in expression levels is an indication that the test tissue is expected to be responsive to KSP inhibitors. If instead expression levels are compared with normal tissue, one concludes that the test tissue will likely respond to KSP treatment if: 1) the expression levels of one or more of those nucleic acids that positively correlate with KSP expression (see, e.g., Tables 1 and 3) are increased, and/or 2) expression levels of one or more of those nucleic acids that negatively correlate with KSP expression (see, e.g., Tables 2 and 4) are decreased.

Other related classification methods involve determining for a tumor sample whether the expression levels of one or more cell cycle genes listed in Tables 1 or 3 are increased and/or whether the expression levels of one or more signal transduction genes from Tables 2 or 4 are decreased. If so, the tumor sample is classified as one that is likely responsive to therapeutic regimes that result in the inhibition of one or more cell cycle genes and/or he activation of one or more signal transduction genes.

C. Diagnostic Methods

Methods for determining presence or absence of certain tumors or cancers in the tissue of a subject are also provided. Such methods initially involve obtaining a test sample from a subject having a tumor or susceptible to development of a tumor. The expression level of one or more of the nucleic acid markers is then determined for the sample. The population of test cells can contain the primary tumor (e.g., the sample is tissue containing the tumor) or can include cells into which the primary tumor has disseminated (e.g., blood or lymphatic fluid).

The expression levels are then compared with the expression levels of the same markers in a control cell population. The status of the control cell population with respect to presence or absence of cancer is known (e.g., the control cell population is from normal tissue, cancerous tissue or a combination of such tissues). So, for example, if the control cell population is representative of normal tissue, then similarity in expression level or expression profile between the test and control cell populations indicates that the test cell population does not contain a tumor or cancerous cells. A difference in expression level or expression profile, in contrast, indicates that the test cells contain a tumor or are cancerous.

If instead the control cell population is representative of tissue with a tumor or cancer, then similarity in expression levels or expression profile means that the test cell population contains a tumor or is cancerous. Alternatively, a difference in expression levels or expression profile indicates that the test cell population is not cancerous or does not contain a tumor.

D. Screening for Candidate Chemotherapeutic Agents

The differentially expressed nucleic acids that are provided can be used in screening methods to identify candidate agents that are useful in treating certain tumors or cancers. These methods generally involve determining whether a candidate agent alters the expression levels for one or more of the markers in a direction that is consistent with a non-cancerous state. Some methods thus involve determining whether the test agent converts an expression profile representative of a cancerous state to an expression profile representative of a non-cancerous state.

The methods initially involve contacting one or more candidate agents with a test cell population. The expression level of one or more of the differentially expressed nucleic acids in the test cell population is then determined. The expression levels in the test cell is next compared with the expression levels for the same nucleic acids in a control cell population that has not been contacted with the therapeutic agent. The cells in both the test cell population and the control cell population typically are selected to be as nearly identical to each other as possible. In this way, differences in expression levels between test and control populations primarily reflect the fact that the test population has been contacted with the candidate agent, whereas the control population has not.

Regardless of whether the control cell population contains only normal cells, cancerous cells or a mixture, the primary inquiry in the comparison is: 1) whether there is a decrease in expression levels for one or more of the nucleic acids that are up-regulated in tumor cells, and 2) whether there is an increase in expression for one or more of those nucleic acids that are down-regulated in tumor cells. A candidate agent having potential chemotherapeutic value is one that decreases expression of one or more nucleic acids that are up-regulated in cancerous cells and/or increases expression of one or more nucleic acids that are down-regulated in cancerous cells.

Some methods optionally involve contacting the test and control cell populations with a carcinogen to induce a cancerous state.

The candidate agent can be any of a number of different types. Exemplary candidate agents include those from natural product libraries, synthetic libraries and random libraries. Often the candidate agents are small molecule compounds (e.g., compounds having a molecular weight of <1000 daltons, or <500 daltons). Examples, include but are not limited to, heterocyclic compounds, urea-based derivatives, β-lactams, oligo-N-substituted glycines, and polycarbamates. Other candidate agents are antisense nucleic acids, ribozymes, or doubled stranded RNAs (see infra). Once a candidate agent has demonstrated potential effectiveness as a chemotherapeutic, it can be tested further to evaluate it's efficacy in preventing tumor growth. Such analyses can be performed utilizing conventional methods for assessing toxicity and clinical effectiveness of chemotherapeutics.

E. Methods to Identify Potential Carcinogens and Methods for Risk Assessment

The differentially expressed nucleic acids that are provided also have value in screening methods designed to identify potential carcinogens. Generally these methods involve determining whether a test agent alters the expression of one or more of the differentially expressed nucleic acids (or an expression profile of these nucleic acids) in a way that is consistent with the expression levels observed for a cancerous state.

A test agent is first contacted with a test cell population (typically a population of normal cells). The test agent is allowed to remain in contact with the test cell population for a sufficiently long period such that the test agent can induce a cancerous state if it has such activity. The test cell population is selected to be capable of expressing the differentially expressed nucleic acids. The expression level of the differentially expressed nucleic acids is then measured and compared with the expression levels of the same nucleic acids in a control cell population that typically has not been contacted with the test agent. The cells in both the test cell population and the control cell population usually are as nearly identical to each other as possible. In this way, differences in expression levels between test and control populations primarily reflect the fact that the test population has been contacted with the test agent, whereas the control population has not.

The comparison involves determining if there is an increase in expression for those differentially expressed nucleic acids that are up-regulated in cancerous tissues and/or if there is a decrease in expression for those differentially expressed genes that are down-regulated in cancerous genes. A test agent that is potentially carcinogenic should cause an increase in expression in the test cell of one or more nucleic acids that are up-regulated in cancerous tissue and/or effect a decrease in expression in the test cell of one or more nucleic acids that are down-regulated in cancerous tissue.

To assess whether a test agent induces formation of a tumor or cancer upon extended exposure or at some point subsequent to exposure, the foregoing method can optionally be extended so that samples are taken from the test cell population at different time points. Thus, certain methods involve multiple sampling from the test population before, during or after initially being contacted with the test agent. For each sample taken, comparison with a reference cell population generally proceeds as just described.

These screening methods can be conducted with essentially any compound that is considered to potentially be carcinogenic. So, for example, the methods can be used to evaluate potential pharmaceuticals, and a variety of non-pharmaceutical compounds, including, but not limited to, solvents, food additives, cosmetic ingredients, cleansers, preservatives, household products, dyes, personal hygiene products, pesticides, herbicides, insecticides and the like.

F. Screening Assays for Compounds that Interact with Target Nucleic Acids

Nucleic acids modulated in cancerous cells can fall into one of several categories, including for example: (1) genes whose modulation leads to tumor or cancer formation; (2) genes whose modulation results in a protective effect against the tumor or cancer formation; or (3) genes that are indicative of a cancer or tumor but that are not directly involved as a causative agent or the cell's protective response.

Target nucleic acids or genes and their respective target gene products are those genes and products shown to affect cancer or tumor formation and thus are not simply markers of a tumor or cancerous state. A variety of assays can be designed to identify compounds that bind to target gene products, bind to other cellular or extracellular proteins that interact with a target gene product, or interfere with the interaction of the target gene product with other cellular or extracellular proteins. For example, the expression level of a target gene product in some instances is reduced and this overall lower level of target gene expression and/or target gene product results in tumor or cancer formation. In such instances, screens can be developed to identify compounds that interact with the target gene or target gene product to increase the expression of the target gene or activity of the target gene product. In so doing, such compounds effectively increase the level of target gene product activity, thereby reducing the likelihood of cancer or tumor formation.

In other instances, up-regulation of a target gene results in increased target gene product that in turn causes tumor or cancer formation. In this instance, screens are designed to identify compounds that interact with the target gene or gene product to decrease the activity of the target gene or gene product. Such compounds can be utilized in treatments to ameliorate the risks of tumors or cancers being formed. The opposite situation also exists in which the up-regulation of a target gene yields a target gene product that exerts a protective effect. The goal of screens in such instances is to identify compounds that enhance the expression of such up-regulated genes or the activity of their gene products, thereby reducing the chance for tumor or cancer formation.

Target genes themselves can be identified by appropriate experiments in which expression of the target gene(s) is artificially modulated independent of exposures that might cause a tissue to become a cancerous. For example, genes whose up-regulation exerts a protective effect can, when cloned, transfected into test cells and expressed at high levels, reduce the likelihood of tumor formation when the cells are challenged with carcinogen. Similarly, for those target genes whose down-regulation exerts a positive effect, deletion of the gene can reduce the risk for tumor or cancer formation. In like manner, the overexpression of target genes whose expression causes tumor or cancer formation can exacerbate the likelihood that a tissue forms a tumor or becomes cancerous, whereas deletion of such a gene can lessen the likelihood for such a response.

1. Assays for Compounds Capable of Binding Target Gene Product

A variety of methods can be developed to identify compounds that bind to a target gene or gene product. In certain assays, the protein encoded by the target gene is contacted with a test compound under suitable conditions for a sufficient period of time to allow the two components to interact and form a complex that can be isolated and/or detected in the reaction mixture. A variety of different formats known to those in the art can be utilized for conducting such binding assays.

For example, either the target gene protein or the test compound can be attached to a solid phase and then the other component added to allow for formation of a test compound/target gene protein complex. Unbound components are removed, typically by washing, under conditions that allow complexes to remain immobilized to the solid support. Detection of complexes can be achieved in various ways. If the non-immobilized component is labeled, complexes can be detected simply by identifying immobilized label on the support. If the non-immobilized component was not labeled prior to complex formation, complexes can be detected using indirect methods. For example, a labeled antibody with binding specificity for the initially non-immobilized component can be added to form a complex with the initially non-immobilized component (alternatively, an unlabeled antibody can be added and than a labeled antibody having binding specificity for the unlabeled antibody added to form a labeled complex).

Binding assays can also be conducted in solution wherein the test compound and target gene protein are allowed to form complexes which can than be separated from uncomplexed components. One such approach includes immobilizing an antibody specific for the target gene product (or less frequently the test compound) which in turn immobilizes the complex to the support. By labeling one of the components immobilized complexes can be detected.

2. Assays for Compounds that Interfere with the Interaction Between Target Gene Products and Other Compounds

In exerting their in vivo effect, target proteins can interact with one or more cellular or extracellular proteins to form complexes. The proteins in such complexes are referred to as binding partners. Compounds capable of disrupting the interaction between such partners can be useful in regulating the activity of the target gene proteins.

Numerous assays can be conducted to disrupt the interaction between the binding partners. One approach involves contacting the target gene product with its binding partner both in the presence and absence of a test compound. The test compound can be included at the time the binding partners are contacted, or can be added sometime subsequent to mixing the binding partners together. Parallel control experiments are conducted under identical conditions, except that the test compound is not included in the control mixture or a control compound known not to influence the binding of the partners is included in the mixture. Formation of complexes between the partners is then detected. The formation of complexes in the control reaction mixture but not in the test mixture indicates that the test compound interferes with the interaction between the binding partners. Such assays can be conducted in heterogeneous assays in which one of the binding members is immobilized to a solid support or in homogeneous assays in which all components are contacted with one another in the liquid phase using methods similar to those set forth in the preceding section.

VII. Therapeutic Treatment Methods

A variety of methods for treating tumors and cancers are also provided. These methods generally involve administering to a subject that has a tumor, or that is susceptible to developing a tumor, a therapeutic agent that modulates expression of one or more of the differentially expressed nucleic acids in an appropriate manner. Both therapeutic and prophylactic methods are provided. In therapeutic methods, a pharmaceutical composition is administered to a subject having or suspected to have a tumor or cancer in an amount sufficient to alleviate one or more symptoms of the tumor or cancer. In some instances, the composition is administered in an amount sufficient to remove the tumor or cause the cancer to go into remission. In prophylactic methods, a pharmaceutical composition is administered to a subject susceptible to, or otherwise at risk for developing a tumor or cancer, in an amount sufficient to reduce or arrest the development of the tumor or cancer. The treatment can be administered in a single dose, but more commonly is administered in several doses.

Because the nucleic acids listed in Tables 1 and 3 are ones whose expression is up-regulated in certain tumors, some methods generally involve administering to the subject an agent that decreases the level of expression of one or more of these nucleic acids and/or inhibiting the activity of the protein they encode. A number of methods known that are known in the art can be utilized to achieve this goal. One approach is to administer an agent (e.g., a nucleic acid) that inhibits expression of the up-regulated genes at either the level of transcription or translation. Examples of such agents include antisense oligonucleotides, ribozymes, triple helix structure and double-stranded RNA (dsRNA), particularly small-interfering RNAs (siRNAs). These agents are discussed in additional detail below. Alternatively, compounds that antagonize the activity of the protein encoded by the up-regulated genes can also be utilized. Examples include antibodies that specifically bind to the encoded protein. Other antagonists are small molecules.

Other treatment methods involve administering an agent that activates the expression of one or more of the nucleic acids listed in Tables 2 or 4 that are down-regulated in certain tumors or cancerous tissue. With this approach, the agent is administered in an amount and for a time sufficient to increase the level of expression of the down-regulated nucleic acid. A variety of agents can be used for this purpose. One option is to administer a nucleic acid that encodes the down-regulated gene product. This nucleic acid is operably linked to appropriate expression control elements to facilitate its expression in the tumor or cancerous tissue. Another option is to administer the protein encoded by the down-regulated nucleic acid or an active fragment thereof directly. Yet another option is to administer an agonist that increases the activity of the protein encoded by the down-regulated gene.

Still other treatment programs involve a combination of the two previous approaches. Such methods thus involve administration of one or more agents to the subject that inhibit the expression of one or more of the up-regulated genes in combination with an agent that promotes expression of one or more of the down-regulated genes.

Regardless of approach, administration can be systemic or local (e.g., proximate to the tumor or cancerous tissue). Further details regarding administration of pharmaceutical compositions are provided infra.

As one example of such methods, KSP inhibitors such as those described in the Background section can be administered to subjects having a tumor is which one or more of the genes listed in Table 1 or 3 are up-regulated and/or one or more of the genes in from Table 2 or 4 or down-regulated, since these genes correlate positively and negatively with KSP expression, respectively.

Similarly, if an analysis shows that a tumor falls into category 1 as described above (i.e., one with high mitotic index), then a compound that inhibits the expression of a cell cycle gene (see, e.g., Table 1) or the activity of the protein it encodes can be administered. Alternatively, or in combination, a compound that activates expression of a signal transduction gene (see, e.g., Table 2) can be administered.

Should an analysis instead demonstrate that the subject has a tumor falling into category 2, then in some instances treatment involves administration of a therapeutic agent that activates expression of a cell cycle gene (see, e.g., Table 1) and/or inhibits the expression of a signal transduction gene (see, e.g., Table 2).

Category 3 tumors can in some cases be treated by administering an therapeutic agent or agents that inhibit one or more cell cycle and signal transduction genes.

The methods and compositions that are provided herein can be utilized to treat a number of different tumors and cancers. Examples of cancers that can be treated include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma [embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma.

Certain methods specifically useful for treating tumors in which KSP levels are increased, such as lung, ovary and breast.

VIII. Compounds for Inhibiting or Enhancing the Synthesis or Activity of Target Genes

A. Activity or Synthesis Inhibition

As discussed above, certain target genes can cause tumor or cancer formation or worsen outcomes associated with such tumors or cancers. The increase in the expression or activity of such target genes and their products can be countered using various methodologies to inhibit the expression, synthesis or activity of such target genes and/or proteins.

For example, antisense, ribozyme, triple helix molecules and antibodies can be utilized to ameliorate the negative effects of such target genes and gene products. Antisense RNA and DNA molecules act directly to block the translation of mRNA by hybridizing to targeted mRNA, thereby blocking protein translation. Hence, a useful target for antisense molecules is the translation initiation region.

Ribozymes are enzymatic RNA molecules that hybridize to specific sequences and then carry out a specific endonucleolytic cleavage reaction. Thus, for effective use, the ribozyme should include sequences that are complementary to the target mRNA, as well as the sequence necessary for carrying the cleavage reaction (see, e.g., U.S. Pat. No. 5,093,246).

Nucleic acids utilized to promote triple helix formation to inhibit transcription are single-stranded and composed of dideoxyribonucleotides. The base composition of such polynucleotides is designed to promote triple helix formation via Hoogsteen base pairing rules and typically require significant stretches of either pyrimidines or purines on one strand of a duplex.

Double stranded RNA (dsRNA) inhibition methods can also be use to inhibit expression of one or more of the differentially expressed nucleic acids. The RNA utilized in such methods is designed such that a least a region of the dsRNA is substantially identical to a region of a differentially expressed nucleic acid (e.g., a target gene); in some instances, the region is 100% identical to the target. For use in mammals, the dsRNA is typically about 19-30 nucleotides in length (i.e., small inhibitory RNAs are utilized (siRNA)). Methods and compositions useful for performing dsRNAi and siRNA are discussed, for example, in PCT Publications WO 98/53083; WO 99/32619; WO 99/53050; WO 00/44914; WO 01/36646; WO 01/75164; WO 02/44321; and published U.S. patent application Ser. No. 10/195,034, each of which is incorporated herein by reference in its entirety for all purposes.

Antibodies having binding specificity for a target gene protein that also interferes with the activity of the gene protein can also be utilized to inhibit gene protein activity. Such antibodies can be generated from full-length proteins or fragments thereof according to the methods described below.

B. Activity Enhancement

Tumor or cancer formation can be exacerbated by under expression of certain target genes and/or by a reduction in activity of a target gene product. Alternatively, the up-regulation of certain target gene products can produce a beneficial effect. In any of these scenarios, it is useful to increase the expression, synthesis or activity of such target genes and proteins.

These goals can be achieved, for example, by increasing the level of target gene product or the concentration of active gene product. In one approach, a target gene protein in the form of a pharmaceutical composition such as that described below is administered to a subject suffering from a tumor or cancer. Alternatively, DNA sequences encoding target gene proteins can be administered to a patient at a concentration sufficient to treat a tumor or cancer or to reduce the risk or a tumor forming. Gene therapy is yet another option and includes inserting one or more copies of a normal target gene, or a fragment thereof capable of producing a functional target protein, into cells using various vectors. Suitable vectors include, for example, adenovirus, adeno-associated virus and retrovirus vectors. Liposomes and other particles capable of introducing DNA into cells can also be utilized in some instances. Cells, typically autologous cells, that express a normal target gene can than be introduced or reintroduced into a patient to treat the tumor or cancer.

X. Antibodies

Antibodies that are immunoreactive with polypeptides expressed from the differentially expressed nucleic acids or fragments thereof are also provided. The antibodies can be polyclonal antibodies, distinct monoclonal antibodies or pooled monoclonal antibodies with different epitopic specificities.

A. Production of Antibodies

The antibodies can be prepared using intact polypeptide or fragments containing antigenic determinants from proteins encoded by differentially expressed genes or target genes as the immunizing antigen. The polypeptide used to immunize an animal can be from natural sources, derived from translated cDNA, or prepared by chemical synthesis. In some instances the polypeptide is conjugated with a carrier protein. Commonly used carriers include keyhole limpet hemocyanin (KLH), thyroglobulin, bovine serum albumin (BSA), and tetanus toxoid. The coupled peptide is then used to immunize the animal (e.g., a mouse, a rat, or a rabbit). Various adjuvants can be utilized to increase the immunological response, depending on the host species and include, but are not limited to, Freund's (complete and incomplete), mineral gels such as aluminum hydroxide, surface active substances such as lysolecithin, pluronic polyols, polyanions, peptides, oil emulsions, dinitrophenol and carrier proteins, as well as human adjuvants such as BCG (bacille Calmette-Guerin) and Corynebacterium parvum.

Monoclonal antibodies can be made from antigen-containing fragments of the protein by the hybridoma technique, for example, of Kohler and Milstein (Nature, 256:495-497, (1975); and U.S. Pat. No. 4,376,110, incorporated by reference in their entirety). See also, Harlow & Lane, Antibodies, A Laboratory Manual (C.S.H.P., NY, 1988), incorporated by reference in its entirety. The antibodies can be of any immunoglobulin class including IgG, IgM, IgE, IgA, IgD and any subclass thereof.

Techniques for generation of human monoclonal antibodies have also been described, including, for example, the human B-cell hybridoma technique (Kosbor et al., Immunology Today 4:72 (1983), incorporated by reference in its entirety); for a review, see also, Larrick et al., U.S. Pat. No. 5,001,065, (incorporated by reference in its entirety). An alternative approach is the generation of humanized antibodies by linking the complementarity-determining regions or CDR regions (see, e.g., Kabat et al., “Sequences of Proteins of Immunological Interest,” U.S. Dept. of Health and Human Services, (1987); and Chothia et al., J. Mol. Biol. 196:901-917 (1987)) of non-human antibodies to human constant regions by recombinant DNA techniques. See Queen et al., Proc. Natl. Acad. Sci. USA 86:10029-10033 (1989) and WO 90/07861 (incorporated by reference in its entirety). Alternatively, one can isolate DNA sequences that encode a human monoclonal antibody or a binding fragment thereof by screening a DNA library from human B cells according to the general protocol set forth by Huse et al., Science 246:1275-1281 (1989) and then cloning and amplifying the sequences which encode the antibody (or binding fragment) of the desired specificity. The protocol described by Huse is rendered more efficient in combination with phage display technology. See, e.g., Dower et al., WO 91/17271 and McCafferty et al., WO 92/01047 (each of which is incorporated by reference). Phage display technology can also be used to mutagenize CDR regions of antibodies previously shown to have affinity for the peptides of the present invention. Antibodies having improved binding affinity are selected.

Techniques developed for the production of “chimeric antibodies” by splicing the genes from a mouse antibody molecule of appropriate antigen specificity together with genes from human antibody molecule of appropriate antigen specificity can be used. A chimeric antibody is a molecule in which different portions are derived from different species, such as those having a variable region derived from a murine monoclonal antibody and a human immunoglobulin constant region. Single chain antibodies specific for the differentially expressed gene products of the invention can be produced according to established methodologies (see, e.g., U.S. Pat. No. 4,946,778; Bird, Science 242:423-426 (1988); Huston et al., Proc. Natl. Acad. Sci. USA 85:5879-5883 (1988); and Ward et al., Nature 334:544-546 (1989), each of which is incorporated by reference in its entirety). Single chain antibodies are formed by linking the heavy and light chain fragments of the Fv region via an amino acid bridge, resulting in a single chain polypeptide.

Antibodies can be further purified, for example, by binding to and elution from a support to which the polypeptide or a peptide to which the antibodies were raised is bound. A variety of other techniques known in the art can also be used to purify polyclonal or monoclonal antibodies (see, e.g., Coligan, et al., Unit 9, Current Protocols in Immunology, Wiley Interscience, (1994), incorporated herein by reference in its entirety).

Anti-idiotype technology can also be utilized in some instances to produce monoclonal antibodies that mimic an epitope. For example, an anti-idiotypic monoclonal antibody made to a first monoclonal antibody will have a binding domain in the hypervariable region that is the “image” of the epitope bound by the first monoclonal antibody.

B. Use of Antibodies

The antibodies that are provided are useful, for example, in screening cDNA expression libraries and for identifying clones containing cDNA inserts which encode structurally-related, immunocrossreactive proteins. See, for example, Aruffo & Seed, Proc. Natl. Acad. Sci. USA 84:8573-8577 (1977) (incorporated by reference in its entirety). Antibodies are also useful to identify and/or purify immunocrossreactive proteins that are structurally related to native polypeptide or to fragments thereof used to generate the antibody. The antibodies can also be used to form antibody arrays to detect proteins expressed by the differentially expressed nucleic acids.

The antibodies can also be used in the detection of differentially expressed genes, such as target and fingerprint gene products. Thus, the antibodies can be used to detect such gene products in specific cells, tissues or serum, for example, and have utility in diagnostic assays. Various diagnostic assays can be utilized, including but not limited to, competitive binding assays, direct or indirect sandwich assays and immunoprecipitation assays (see, e.g., Monoclonal Antibodies: A Manual of Techniques, CRC Press, Inc. (1987) pp. 147-158). When utilized in diagnostic assays, the antibodies are typically labeled with a detectable moiety. The label can be any molecule capable of producing, either directly or indirectly, a detectable signal. Suitable labels include, for example, radioisotopes (e.g., ³H, ¹⁴C, ³²P, ³⁵S, ¹²⁵I), fluorophores (e.g., fluorescein and rhodamine dyes and derivatives thereof), chromophores, chemiluminescent molecules, an enzyme substrate (including the enzymes luciferase, alkaline phosphatase, beta-galactosidase and horse radish peroxidase, for example). The antibodies can also be utilized in the development of antibody arrays.

As noted above, antibodies are useful in inhibiting the expression products of the differentially expressed nucleic acids and are valuable in inhibiting the action of certain target gene products (e.g., target gene products identified as causing or exacerbating tumor or cancer formation). Hence, the antibodies also find utility in a variety of therapeutic applications.

XI. Pharmaceutical Compositions

Compounds identified during the various screening methods that either inhibit or enhance the activity of differentially expressed gene products such as target genes products can be formulated into pharmaceutical compositions for therapeutic use. For example, compounds that inhibit target gene products associated with tumor formation (e.g., antibodies, antisense sequences, ribozymes, triple helix molecules) can be utilized in preparing pharmaceutical compositions. Alternatively, compounds identified during screening that enhance the concentration or activity of target gene products that exert a positive effect can be incorporated into pharmaceutical compositions.

A. Composition

The pharmaceutical compositions used for treatment of cancers and tumors comprise an active ingredient such as the inhibitory or activity-enhancing compounds such as described herein and, optionally, various other components.

Thus, for example, the compositions can also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents, detergents and the like.

The composition can also include any of a variety of stabilizing agents, such as an antioxidant for example. When the pharmaceutical composition includes a polypeptide, the polypeptide can be complexed with various well known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include the production of sulfate, gluconate, citrate, phosphate and the like. The polypeptides of the composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

B. Dosage

The pharmaceutical compositions can be administered for prophylactic and/or therapeutic treatments. The active ingredient in the pharmaceutical compositions typically is present in a therapeutic amount, which is an amount sufficient to slow or reverse tumor formation, to eliminate the tumor, or to remedy symptoms associated with the tumor or cancer. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized.

In prophylactic applications, compositions containing the compounds that are provided are administered to a patient susceptible to or otherwise at risk of tumor formation. Such an amount is defined to be a “prophylactically effective” amount or dose. In this use, the precise amounts depends on the patient's state of health and weight. Typically, the dose ranges from about 1 to 500 mg of purified protein per kilogram of body weight, with dosages of from about 5 to 100 mg per kilogram being more commonly utilized.

C. Administration

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Suitable formulations for rectal administration include, for example, suppositories, which consist of the packaged active ingredient with a suppository base. Suitable suppository bases include natural or synthetic triglycerides or paraffin hydrocarbons. In addition, it is also possible to use gelatin rectal capsules which consist of a combination of the packaged nucleic acid with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Formulations for injection can be presented in unit dosage form, e.g., in ampules or in multidose containers, with an added preservative. The compositions are formulated as sterile, substantially isotonic and in full compliance with all Good Manufacturing Practice (GMP) regulations of the U.S. Food and Drug Administration.

XII. Methods for Identifying Gene Expression Changes

A. Nucleic Acid Detection

Gene expression changes can be monitored at the nucleic acid level by a variety of methods known in the art including, for example, differential display PCR, probe array methods, quantitative reverse transcriptase (RT)-PCR, Northern analysis, subtractive hybridization, GENECALLING™, RNase protection, serial analysis of gene expression (SAGE), and in situ assays. Most methods begin with the isolation of RNA (typically mRNA) from a sample and then determination of the level of expression of genes of interest.

1. mRNA Isolation

To measure the transcription level (and thereby the expression level) of a gene or genes, a nucleic acid sample comprising mRNA transcript(s) of the gene(s) or gene fragments, or nucleic acids derived from the mRNA transcript(s) is obtained. A nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, are all derived from the mRNA transcript and detection of such derived products is indicative of the presence and/or abundance of the original transcript in a sample. Thus, suitable samples include, but are not limited to, mRNA transcripts of the gene or genes, cDNA reverse transcribed from the mRNA, cRNA transcribed from the cDNA, DNA amplified from the genes, RNA transcribed from amplified DNA.

In some methods, a nucleic acid sample is the total mRNA isolated from a biological sample; in other instances, the nucleic acid sample is the total RNA from a biological sample. The term “biological sample” or simply “sample”, as used herein, refers to a sample obtained from an organism or from components of an organism, such as cells, biological tissues and fluids. In some methods, the sample is from a human patient. Such samples include sputum, blood, blood cells (e.g., white cells), tissue or fine needle biopsy samples, urine, peritoneal fluid, and fleural fluid, or cells therefrom. Biological samples can also include sections of tissues such as frozen sections taken for histological purposes. Often two samples are provided for purposes of comparison. The samples can be, for example, from different cell or tissue types, from different individuals or from the same original sample subjected to two different treatments (e.g., drug-treated and control).

Any RNA isolation technique that does not select against the isolation of mRNA can be utilized for the purification of such RNA samples. For example, methods of isolation and purification of nucleic acids are described in detail in WO 97/10365, WO 97/27317, Chapter 3 of Laboratory Techniques in Biochemistry and Molecular Biology: Hybridization With Nucleic Acid Probes, Part I. Theory and Nucleic Acid Preparation, (P. Tijssen, ed.) Elsevier, N.Y. (1993); Chapter 3 of Laboratory Techniques in Biochemistry and Molecular Biology: Hybridization With Nucleic Acid Probes, Part 1. Theory and Nucleic Acid Preparation, (P. Tijssen, ed.) Elsevier, N.Y. (1993); and Sambrook et al., Molecular Cloning: A Laboratory Manual, Cold Spring Harbor Press, N.Y., (1989); Current Protocols in Molecular Biology, (Ausubel, F. M. et al., eds.) John Wiley & Sons, Inc., New York (1987-1993). Large numbers of tissue samples can be readily processed using techniques known in the art, including, for example, the single-step RNA isolation process of Chomczynski, P. described in U.S. Pat. No. 4,843,155.

2. Differential Display PCR

Differential display PCR (DD PCR) is one method that is useful for identifying genes that have been differentially expressed under different sets of conditions. DD PCR utilizes a modification of the well-established PCR technique (see, e.g., U.S. Pat. Nos. 4,683,202 and 4,683,195) in which a primer pair consisting of a primer that hybridizes to the poly A tail of the mRNA and an arbitrary primer is used to amplify various segments of the mRNAs contained within a sample. The resulting amplification products are separated on a sequencing gel. Comparison of bands on separate gels obtained for test and control samples allows for the identification of differentially expressed genes. Bands that are differentially expressed can be excised and analyzed further to determine the identity of the differentially expressed gene.

DD-PCR has an advantage relative to certain other methods of differential gene expression detection in that no prior knowledge of gene sequences is required. Further, because the PCR conditions are conducted under relatively low stringency conditions such that only 5-6 bases at the 3′ end of each primer need match a potential template, with a sufficient number of primers it is possible to detect most expressed genes.

Further guidance regarding the use of DD PCR can be found in a number of sources including, for example, U.S. Pat. Nos. 5,262,311; 5,599,672; and Liang, P. and Pardee, A. B., Science 257:967-971 (1992); Liang, P., et al., Methods of Enzymol. 254:304-321 (1995); Liang, P. et al., Nucl. Acids Res. 22:5763-5764 (1994); Liang, P. and Pardee, A. B., Curr. Opin. in Immunology 7:274-280 (1995); and Reeves, S. A., et al., BioTechniques 18:18-20 (1995), each of which is incorporated by reference in its entirety.

3. Probe Arrays

Array-based expression monitoring is another useful approach for detecting differential gene expression. This approach can be used to achieve high throughput analysis. The arrays utilized in differential gene expression analysis can be of a variety of differing types, depending in part upon whether the gene and/or gene fragments to be detected are known in advance of an experiment. For example, some arrays contain short polynucleotide probes, while other arrays contain full-length cDNAs. Regardless of the nature of the probe, the probes are typically attached to some type of support.

In probe array methods, once nucleic acids have been obtained from a test sample, they typically are reversed transcribed into labeled cDNA, although labeled mRNA can be used directly. The test sample containing the labeled nucleic acids is then contacted with the probes of the array. After allowing a period for targets to hybridize to the probes, the array is typically subjected to one or more high stringency washes to remove unbound target and to minimize nonspecific binding to the nucleic acid probes of the arrays. Binding of target nucleic acid, and thus detection of expressed genes in the sample, is detected using any of a variety of commercially available scanners and accompanying software programs.

General methods for using expression arrays are described in WO 97/10365, PCT/US/96/143839 and WO 97/27317, each of which are incorporated by reference in their entirety. Additional discussion regarding the use of microarrays in expression analysis can be found, for example, in Duggan, et al., Nature Genetics Supplement 21:10-14 (1999); Bowtell, Nature Genetics Supplement 21:25-32 (1999); Brown and Botstein, Nature Genetics Supplement 21:33-37 (1999); Cole et al., Nature Genetics Supplement 21:38-41 (1999); Debouck and Goodfellow, Nature Genetics Supplement 21:48-50 (1999); Bassett, Jr., et al., Nature Genetics Supplement 21:51-55 (1999); and Chakravarti, Nature Genetics Supplement 21:56-60 (1999), each of which is incorporated herein by reference in its entirety.

The probes utilized in the arrays of the present invention can include, for example, synthesized probes of relatively short length (e.g., a 20-mer or a 25-mer), cDNA (full length or fragments of gene), amplified DNA, fragments of DNA (generated by restriction enzymes, for example) and reverse transcribed DNA. For a review on different types of microarrays, see for example, Southern et al., Nature Genetics Supplement 21:5-9 (1999), which is incorporated herein by reference.

After hybridization of control and target samples to an array containing one or more probe sets as described above and optional washing to remove unbound and nonspecifically bound probe, the hybridization intensity for the respective samples is determined for each probe in the array. For fluorescent labels, hybridization intensity can be determined by, for example, a scanning confocal microscope in photon counting mode. Appropriate scanning devices are described by e.g., U.S. Pat. No. 5,578,832 to Trulson et al., and U.S. Pat. No. 5,631,734 to Stern et al. (both of which are incorporated by reference in their entirety) and are available from Affymetrix, Inc., under the GeneChip™ label. Some types of label provide a signal that can be amplified by enzymatic methods (see Broude, et al., Proc. Natl. Acad. Sci. U.S.A. 91, 3072-3076 (1994)). A variety of other labels are also suitable including, for example, radioisotopes, chromophores, magnetic particles and electron dense particles.

The position of label can be detected for each probe in the array using a reader, such as described by U.S. Pat. No. 5,143,854, WO 90/15070, and Trulson et al., U.S. Pat. No. 5,578,832, each of which is incorporated by reference in its entirety. For customized arrays, the hybridization pattern can then be analyzed to determine the presence and/or relative amounts or absolute amounts of known mRNA species in samples being analyzed as described in e.g., WO 97/16365. Comparison of the expression patterns of two samples is useful for identifying mRNAs and their corresponding genes that are differentially expressed between the two samples.

The quantitative monitoring of expression levels for large numbers of genes can prove valuable in elucidating gene function, exploring the mechanism(s) associated with a tumor, and for the discovery of potential therapeutic and diagnostic targets and methods.

4. Quantitative RT-PCR

A variety of so-called “real time amplification” methods or “real time quantitative PCR” methods can also be utilized to determine the quantity of mRNA present in a sample by measuring the amount of amplification product formed during an amplification process. Fluorogenic nuclease assays are one specific example of a real time quantitative method that can be used successfully with the methods of the present invention (see Example 2). The basis for this method of monitoring the formation of amplification product is to measure continuously PCR product accumulation using a dual-labeled fluorogenic oligonucleotide probe—an approach frequently referred to in the literature simply as the “TaqMan” method.

The probe used in such assays is typically a short (ca. 20-25 bases) polynucleotide that is labeled with two different fluorescent dyes. The 5′ terminus of the probe is typically attached to a reporter dye and the 3′ terminus is attached to a quenching dye, although the dyes could be attached at other locations on the probe as well. The probe is designed to have at least substantial sequence complementarity with the probe binding site. Upstream and downstream PCR primers that bind to flanking regions of the locus are also added to the reaction mixture.

When the probe is intact, energy transfer between the two fluorophors occurs and the quencher quenches emission from the reporter. During the extension phase of PCR, the probe is cleaved by the 5′ nuclease activity of a nucleic acid polymerase such as Taq polymerase, thereby releasing the reporter from the polynucleotide-quencher and resulting in an increase of reporter emission intensity which can be measured by an appropriate detector.

One detector which is specifically adapted for measuring fluorescence emissions such as those created during a fluorogenic assay is the ABI 7700 manufactured by Applied Biosystems, Inc. in Poster City, Calif. Computer software provided with the instrument is capable of recording the fluorescence intensity of reporter and quencher over the course of the amplification. These recorded values can then be used to calculate the increase in normalized reporter emission intensity on a continuous basis and ultimately quantify the amount of the mRNA being amplified.

Additional details regarding the theory and operation of fluorogenic methods for making real time determinations of the concentration of amplification products are described, for example, in U.S. Pat. No. 5,210,015 to Gelfand, U.S. Pat. No. 5,538,848 to Livak, et al., and U.S. Pat. No. 5,863,736 to Haaland, as well as Heid, C. A., et al., Genome Research, 6:986-994 (1996); Gibson, U. E. M, et al., Genome Research 6:995-1001 (1996); Holland, P. M., et al., Proc. Natl. Acad. Sci. USA 88:7276-7280, (1991); and Livak, K. J., et al., PCR Methods and Applications 357-362 (1995), each of which is incorporated by reference in its entirety.

5. Dot Blot Assays

Another option for detecting differential gene expression includes spotting a solution containing a nucleic acid known to be differentially expressed on a support. Spotting can be performed robotically to increase reproducibility using an instrument such as the BIODOT instrument manufactured by Cartesian Technologies, Inc., for example. The nucleic acids are typically attached to the support using UV cross-linking methods that are known in the art. Labeled cDNA clones prepared from a mRNA sample of interest are treated to remove self-annealing or annealing between different clones and then contacted with the nucleic acids bound to the support and allowed sufficient time to hybridize with the nucleic acids on the support. Supports are washed to remove unhybridized clones. The formation of hybridized complexes can be detected using various known techniques including, for example, exposing a phosphor screen and subsequent scanning using a phosphorimager (e.g., such as available from Molecular Dynamics). This method can be repeated with mRNA obtained from test cells from tumors and control cells from normal tissue to identify genes that are differentially expressed. As described further in Example 1, such methods were utilized in the present invention to confirm the results obtained by DD PCR. For further guidance on such methods, see, e.g., Sambrook, et al., Molecular Cloning: A Laboratory Manual, 2nd ed., Cold Spring Harbor Laboratory Press (1989).

6. Subtractive Hybridization

This approach typically includes isolating mRNA from two different sources (e.g., a test cell from a tumor and a control cell from normal tissue). The isolated mRNA from one of the sources is typically reverse-transcribed to form a labeled cDNA. The resulting single-stranded is hybridized to a large excess of mRNA from the second closely related cell. After hybridization, the cDNA:mRNA hybrids are removed using standard techniques. The remaining “subtracted” labeled cDNA can then be used to screen a cDNA or genomic library of the same cell population to identify those genes that are potentially differentially expressed. See, for example, Sargent, T. D., Meth. Enzymol. 152:423-432 (1987); and Lee et al., Proc. Natl. Acad. Sci. USA, 88:2825-2830 (1991).

7. In Situ Hybridization

This approach involves the in situ hybridization of labeled probes to one or more of the differentially expressed genes of interest. Because the method is performed in situ, it has the advantage that it is not necessary to prepare RNA from the cells. The method involves initially fixing test cells to a support (e.g., the walls of a microtiter well) and then permeabilizing the cells with an appropriate permeabilizing solution. A solution containing the labeled probes is then contacted with the cells and the probes allowed to hybridize with the complementary differentially expressed genes. Excess probe is digested, washed away and the amount of hybridized probe measured. See, e.g., Harris, D. W., Anal. Biochem. 243:249-256 (1996); Singer, et al., Biotechniques 4:230-250 (1986); Haase et al., Methods in Virology, vol. VII, pp. 189-226 (1984); and Nucleic Acid Hybridization: A Practical Approach (Hames, et al., Eds.), (1987), each of which is incorporated by reference in its entirety.

8. Differential Screening

This technique involves the duplicate screening of a cDNA library in which one copy of the library is screened with a total cell cDNA probe corresponding to the mRNA population of one cell type. The duplicate copy of the cDNA library is screened with a total cDNA probe corresponding to the mRNA population of the second cell type. For instance, one cDNA probe corresponds to the total cell cDNA probe of a cell obtained from a control subject. The second cDNA probe corresponds to the total cell cDNA probe of the same cell type obtained from a subject having a tumor. Clones that hybridize to one probe but not the other potentially represent clones derived from differentially expressed genes. Such methods are described, for example, by Tedder, T. F., et al., Proc. Natl. Acad. Sci. USA 85:208-212 (1988).

9. Other Miscellaneous Methods

Several recently developed methods can also be used to detect differentially expressed genes. These include the GENECALLING™ method (see, e.g., U.S. Pat. No. 5,871,697; and Shimikets et al., Nature Biotechnology 17:798-803 (1999), each incorporated herein by reference), and the Serial Analysis of Gene Expression (SAGE) method (see, e.g., U.S. Pat. No. 5,866,330; Velculescu et al. (1995) Science 270:484-487; and Zhang et al. (1997) Science 276:1268-1272, each incorporated herein by reference).

B. Protein Detection

Expression levels can be determined by detecting the level at which a protein encoded by a differentially expressed nucleic acid is present in a sample. A number of methods for detecting proteins in a sample are known in the art, including Western blots and immunohistochemical staining, for example. Immunohistochemical staining methods typically first involve dehydrating and fixing a tissue sample. The sample is then labeled with labeled antibodies that specifically bind to the protein encoded by a differehtially expressed nucleic acid. Antibodies of any of the types described in the definition section can be used. Methods for preparing suitable antibodies are described above. The label can be directly attached to the antibody or to a secondary antibody that binds to the primary antibody. The level of expression of the protein can be comparing stain intensities with a control or by counting labeled cells, for example.

XIII. Devices for Detecting Differentially Expressed Nucleic Acids

A. Customized Probe Arrays

1. Probes for Target Nucleic Acids

The differentially expressed nucleic acids that are provided can be utilized to prepare custom probe arrays for use in screening and diagnostic applications. In general, such arrays include probes such as those described above in the section on differentially expressed nucleic acids, and thus include probes complementary to full-length differentially expressed nucleic acids (e.g., cDNA arrays) and shorter probes that are typically 10-30 nucleotides long (e.g., synthesized arrays). Typically, the arrays include probes capable of detecting a plurality of the differentially expressed nucleic acids of the invention. For example, such arrays generally include probes for detecting at least 2, 3, 4, 5, 6, 7, 8, 9 or 10 differentially expressed nucleic acids. For more complete analysis, the arrays can include probes for detecting at least 12, 14, 16, 18 or 20 differentially expressed nucleic acids. In still other instances, the arrays include probes for detecting at least 25, 30, 35, 40, 45 or all the differentially expressed nucleic acids that are identified herein.

2. Control Probes

(a) Normalization Controls

Normalization control probes are typically perfectly complementary to one or more labeled reference polynucleotides that are added to the nucleic acid sample. The signals obtained from the normalization controls after hybridization provide a control for variations in hybridization conditions, label intensity, reading and analyzing efficiency and other factors that can cause the signal of a perfect hybridization to vary between arrays. Signals (e.g., fluorescence intensity) read from all other probes in the array can be divided by the signal (e.g., fluorescence intensity) from the control probes thereby normalizing the measurements.

Virtually any probe can serve as a normalization control. However, hybridization efficiency can vary with base composition and probe length. Normalization probes can be selected to reflect the average length of the other probes present in the array, however, they can also be selected to cover a range of lengths. The normalization control(s) can also be selected to reflect the (average) base composition of the other probes in the array. Normalization probes can be localized at any position in the array or at multiple positions throughout the array to control for spatial variation in hybridization efficiently.

(b) Mismatch Controls

Mismatch control probes can also be provided; such probes function as expression level controls or for normalization controls. Mismatch control probes are typically employed in customized arrays containing probes matched to known mRNA species. For example, certain arrays contain a mismatch probe corresponding to each match probe. The mismatch probe is the same as its corresponding match probe except for at least one position of mismatch. A mismatched base is a base selected so that it is not complementary to the corresponding base in the target sequence to which the probe can otherwise specifically hybridize. One or more mismatches are selected such that under appropriate hybridization conditions (e.g. stringent conditions) the test or control probe can be expected to hybridize with its target sequence, but the mismatch probe cannot hybridize (or can hybridize to a significantly lesser extent). Mismatch probes can contain a central mismatch. Thus, for example, where a probe is a 20 mer, a corresponding mismatch probe can have the identical sequence except for a single base mismatch (e.g., substituting a G, a C or a T for an A) at any of positions 6 through 14 (the central mismatch).

(c) Sample Preparation, Amplification, and Quantitation Controls

Arrays can also include sample preparation/amplification control probes. Such probes can be complementary to subsequences of control genes selected because they do not normally occur in the nucleic acids of the particular biological sample being assayed. Suitable sample preparation/amplification control probes can include, for example, probes to bacterial genes (e.g., Bio B) where the sample in question is a biological sample from a eukaryote.

The RNA sample can then be spiked with a known amount of the nucleic acid to which the sample preparation/amplification control probe is complementary before processing. Quantification of the hybridization of the sample preparation/amplification control probe provides a measure of alteration in the abundance of the nucleic acids caused by processing steps. Quantitation controls are similar. Typically, such controls involve combining a control nucleic acid with the sample nucleic acid(s) in a known amount prior to hybridization. They are useful to provide a quantitative reference and permit determination of a standard curve for quantifying hybridization amounts (concentrations).

3. Array Synthesis

Nucleic acid arrays for use in the present invention can be prepared in two general ways. One approach involves binding DNA from genomic or cDNA libraries to some type of solid support, such as glass for example. (See, e.g., Meier-Ewart, et al., Nature 361:375-376 (1993); Nguyen, C. et al., Genomics 29:207-216 (1995); Zhao, N. et al., Gene, 158:207-213 (1995); Takahashi, N., et al., Gene 164:219-227 (1995); Schena, et al., Science 270:467-470 (1995); Southern et al., Nature Genetics Supplement 21:5-9 (1999); and Cheung, et al., Nature Genetics Supplement 21:15-19 (1999), each of which is incorporated herein in its entirety for all purposes.)

The second general approach involves the synthesis of nucleic acid probes. One method involves synthesis of the probes according to standard automated techniques and then post-synthetic attachment of the probes to a support. See for example, Beaucage, Tetrahedron Lett., 22:1859-1862 (1981) and Needham-VanDevanter, et al., Nucleic Acids Res., 12:6159-6168 (1984), each of which is incorporated herein by reference in its entirety. A second broad category is the so-called “spatially directed” polynucleotide synthesis approach. Methods falling within this category further include, by way of illustration and not limitation, light-directed polynucleotide synthesis, microlithography, application by ink jet, microchannel deposition to specific locations and sequestration by physical barriers.

Light-directed combinatorial methods for preparing nucleic acid probes are described in U.S. Pat. Nos. 5,143,854 and 5,424,186 and 5,744,305; PCT patent publication Nos. WO 90/15070 and 92/10092; EP 476,014; Fodor et al., Science 251:767-777 (1991); Fodor, et al., Nature 364:555-556 (1993); and Lipshutz, et al., Nature Genetics Supplement 21:20-24 (1999), each of which is incorporated herein by reference in its entirety. These methods entail the use of light to direct the synthesis of polynucleotide probes in high-density, miniaturized arrays. Algorithms for the design of masks to reduce the number of synthesis cycles are described by Hubbel et al., U.S. Pat. No. 5,571,639 and U.S. Pat. No. 5,593,839, and by, Fodor et al., Science 251:767-777 (1991), each of which is incorporated herein by reference in its entirety.

Other combinatorial methods that can be used to prepare arrays for use in the current invention include spotting reagents on the support using ink jet printers. See Pease et al., EP 728, 520, and Blanchard, et al. Biosensors and Bioelectronics II: 687-690 (1996), which are incorporated herein by reference in their entirety. Arrays can also be synthesized utilizing combinatorial chemistry by utilizing mechanically constrained flowpaths or microchannels to deliver monomers to cells of a support. See Winkler et al., EP 624,059; WO 93/09668; and U.S. Pat. No. 5,885,837, each of which is incorporated herein by reference in its entirety.

4. Array Supports

Supports can be made of any of a number of materials that are capable of supporting a plurality of probes and compatible with the stringency wash solutions, Examples of suitable materials include, for example, glass, silica, plastic, nylon or nitrocellulose. Supports are generally are rigid and have a planar surface. Supports typically have from 1-10,000,000 discrete spatially addressable regions, or cells. Supports having 10-1,000,000 or 100-100,000 or 1000-100,000 regions are common. The density of cells is typically at least 1000, 10,000, 100,000 or 1,000,000 regions within a square centimeter. Each cell includes at least one probe; more frequently, the various cells include multiple probes. In general each cell contains a single type of probe, at least to the degree of purity obtainable by synthesis methods, although in other instances some or all of the cells include different types of probes. Further description of array design is set forth in WO 95/11995, EP 717,113 and WO 97/29212, which are incorporated by reference in their entirety.

XIII. Kits

Kits containing components necessary to conduct the screening and diagnostic methods of the invention are also provided. Some kits typically include a plurality of probes that hybridize under stringent conditions to the different differentially expressed nucleic acids that are provided. Other kits include a plurality of different primer pairs, each pair selected to effectively prime the amplification of a different differentially expressed nucleic acid. In the case when the kit includes probes for use in quantitative RT-PCR, the probes can be labeled with the requisite donor and acceptor dyes, or these can be included in the kit as separate components for use in preparing labeled probes.

The kits can also include enzymes for conducting amplification reactions such as various polymerases (e.g., RT and Taq), as well as deoxynucleotides and buffers. Cells capable of expressing one or more of the differentially expressed nucleic acids of the invention can also be included in certain kits.

Typically, the different components of the kit are stored in separate containers. Instructions for use of the components to conduct an analysis are also generally included.

The following examples are offered to illustrate certain aspects of the methods and devices that are provided; it should be understood that these examples are not to be construed to limit the claimed invention.

### EXAMPLE 1

### Identification of Differentially Expressed Genes

A. Analysis of KSP Expression in Various Tissues

A Gene Logic database containing a collection of gene expression profiles of pathologically “normal” and diseased human tissues was used to identify normal organs that express relatively high levels of KSP. A majority of the tissues within the database are derived from malignant tumors and surrounding normal tissues (used as normal profiles) and also contains extensive clinical histories on each tissue.

FIG. 1 shows the expression of KSP across a panel of “normal” tissues. These results show that KSP expression is not ubiquitous. Highest levels of KSP expression are seen in proliferative tissues such as thymus and bone marrow, with moderate expression in organs of the digestive tract such as colon, duodenum, esophagus, stomach and small intestine. The finding that KSP is expressed at relatively high levels in tissue that undergoes comparatively high levels of cellular proliferation is consistent with the role of KSP in mitosis.

B. KSP Expression in Tumors and Normal Tissue

Next, the database was queried to identify tumors that over express KSP with respect to surrounding “normal” tissues. Upon evaluating tumors that over express KSP, it was observed that there is no one particular tumor type that shows increased expression of KSP with respect to normal tissue expression. As illustrated in FIG. 2, the trend of KSP expression in tumors is generally higher than normal tissues, yet there are certain tumors that exhibit “normal” expression of KSP. Hence, tumors can essentially be divided into two categories based on KSP expression: those that exhibit “normal” expression of KSP and those that exhibit “high” expression of KSP with respect to normal tissue expression (i.e., tumors in which KSP expression is up-regulated).

C. Identification of Genes that Positively and Negatively Correlate with KSP Expression

To determine whether differences in gene expression could account for the biological differences between these two classes of tumors, multivariate analysis of gene expression data was performed using unsupervised learning techniques such as Principal Component Analysis (PCA) and Hierarchical clustering, as well as supervised learning techniques such as Partial Least Square-Discriminant Analysis (PLS-DA).

1. Nucleic Acid Probe Array

The Human U133 chip set (A and B chips) from Affymetrix represents approximately 44000 gene probes which constitute all of the known genes, as well as a large number of EST (Expressed Sequence Tag) sequences of unknown function. These are in-situ synthesized oligonucleotide arrays that bind to cRNA probes that represent the abundance of transcript within a given sample. The MAS5.0 software is used to normalize and analyze data across multiple chips. The Gene Logic database contains pre-normalized intensities for each chip.

2. Data Set Filtering

Prior to analysis, all samples within Breast Malignant and Breast Normal tissues were checked for RNA quality by assessing the 3′:5′ ratios. A recommended cutoff of a ratio of 3 was used to eliminate samples that had poor RNA quality. Since the pathologically “normal” samples are isolated from surrounding “normal” tissue of malignant tumors, a second quality control step was implemented to eliminate gene expression profiles of “normal” samples that appear to cluster with the malignant tissues. This could arise from contaminating malignant tissues that alter gene expression data to look more like malignant tissues than normal. Principal Component Analysis (PCA) was applied on log (10) transformed intensities from all 44,000 genes across 74 “normal” and 400 malignant breast tissues to identify outliers that cluster with malignant tissues. Principal component analysis is a decomposition technique that uses variability in gene expression data to identify the most significant themes or patterns of expression within a data set. The most abundant variability is displayed as the first and second principal components which are eigenvectors induced by linear transformation of the data to generate eigenvalues. Eigenvectors of the largest eigenvalues are represented in the first principal component. PCA as well as graphical visualization of data was performed using the SIMCA-P 9.0, Umetrix, Sweden. Using PCA, a total of 51 “normal” tissues were selected as representing “normal” gene expression.

3. Data Analysis

Using an intensity cutoff of 70 (see FIG. 2), the breast infiltrating carcinomas were divided into two classes: Class 1 which contained expression profiles of tumors that showed “normal” expression of KSP, and Class 2 which contained expression profiles of tumors that exhibited “high” expression of KSP.

PCA of these tumor samples using all 44,000 probes shows that these tumors separate into three classes, suggesting that there may be distinctly different underlying biological processes that drive these tumors to progress. A supervised learning algorithm, Partial Least Squares-Discriminant Analysis (PLS-DA) was used to identify the genes that are most significantly responsible for this separation. PLS-DA is called a supervised learning method because in this case, qualitative variables are made (two classes) and the algorithm is asked to use the quantitative variables (gene expression data) to determine what the major variables are between the subjective classes. This is unlike PCA, where no a priori knowledge is used to drive separation. SIMCA-P 9.0 was used to perform PLS-DA and visualize the results. Data was log transformed and scaled to Unit Variance (weight computed as 1/Std deviation). Using PLS-DA, variables of importance scores (VIP) were given to each gene of the 44,000 based on significance of contribution to the separation. Hierarchical clustering was then used on the 169 most significant genes to identify distinct patterns of gene expression that are different between the two classes of cancers.

FIG. 3 shows the results of this analysis for 200 different tissue samples. In this diagram, the 200 different tumor tissue samples (individuals) are represented along the x-axis. As indicated, the left-hand side of the diagram represents results for individuals whose tissue samples had normal levels of KSP; the right-hand side are results for individuals with elevated KSP levels. As indicated, the cluster analysis diagram can be divided into six regions. Regions A, B and C include genes that are primarily signal transduction genes (see Table 2), but also include genes from other families such as listed in Table 4. Regions D, E and F generally correspond to genes that fall within the class of cell cycle genes (see Table 1). Genes that are up-regulated are shown as dark spots; whereas, genes that are down-regulated are shown as light-colored spots.

As can be seen, the tumors fall into three general classes. Tumors with normal KSP levels showed significant up-regulation of signal transduction genes (region A), but significant down-regulation of cell cycle genes (region D). Most tumors with high levels of KSP, in contrast, exhibited down-regulation of signal transduction genes (region B) and up-regulation of cell cycle genes (region E). But a third group of tumors from those having high KSP levels, showed up-regulation of both signal transduction genes (region C) and cell cycle genes (region F). Those genes whose expression correlates positively with KSP expression are listed in Table 1; those genes that correlate negatively are listed in Table 2.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes to the same extent as if each individual publication, patent or patent application were specifically and individually indicated to be so incorporated by reference.

TABLE 1
Genes That Positively Correlate With KSP Expression

| Differential | | GenBank | | |
|---|---|---|---|---|
| Gene No. | Clone_ID | Accession No. | Locus Link | NAME |
| 1 | 204244_s_at | NM_006716.1 | LL: 10926 | activator of S phase kinase |
| 2 | 212021_s_at | BF001806 | LL: 4288 | antigen identified by monoclonal antibody Ki-67 |
| 3 | 202094_at | AA648913 | LL: 332 | baculoviral IAP repeat-containing 5 (survivin) |
| 4 | 209642_at | AF043294.2 | LL: 699 | BUB1 budding uninhibited by benzimidazoles |
| | | | | 1 homolog (yeast) |
| 5 | 202870_s_at | NM_001255.1 | LL: 991 | CDC20 cell division cycle 20 homolog (S. cerevisiae) |
| 6 | 201897_s_at | NM_001826.1 | LL: 1163 | CDC28 protein kinase 1 |
| 7 | 204170_s_at | NM_001827.1 | LL: 1164 | CDC28 protein kinase 2 |
| 8 | 204126_s_at | NM_003504.1 | LL: 8318 | CDC45 cell division cycle 45-like (S. cerevisiae) |
| 9 | 203213_at | AL524035 | LL: 983 | cell division cycle 2, G1 to S and G2 to M |
| 10 | 204695_at | AI343459 | LL: 993 | cell division cycle 25A |
| 11 | 205167_s_at | NM_001790.2 | LL: 995 | cell division cycle 25C |
| 12 | 204962_s_at | NM_001809.2 | LL: 1058 | centromere protein A (17 kD) |
| 13 | 205046_at | NM_001813.1 | LL: 1062 | centromere protein E (312 kD) |
| 14 | 207828_s_at | NM_005196.1 | LL: 1063 | centromere protein F (350/400 kD, mitosin) |
| 15 | 208696_at | AF275798.1 | LL: 22948 | chaperonin containing TCP1, subunit 5 (epsilon) |
| 16 | 205394_at | NM_001274.1 | LL: 1111 | CHK1 checkpoint homolog (S. pombe) |
| 17 | 204775_at | NM_005441.1 | LL: 8208 | chromatin assembly factor 1, subunit B (p60) |
| 18 | 210052_s_at | AF098158.1 | LL: 22974 | chromosome 20 open reading frame 1 |
| 19 | 218663_at | NM_022346.1 | LL: 64151 | chromosome condensation protein G |
| 20 | 203418_at | NM_001237.1 | LL: 890 | cyclin A2 |
| 21 | 214710_s_at | BE407516 | LL: 891 | cyclin B1 |
| 22 | 202705_at | NM_004701.2 | LL: 9133 | cyclin B2 |
| 23 | 205034_at | NM_004702.1 | LL: 9134 | cyclin E2 |
| 24 | 209714_s_at | AF213033.1 | LL: 1033 | cyclin-dependent kinase inhibitor 3 (CDK2-associated |
| | | | | dual specificity phosphatase) |
| 25 | 48808_at | AI144299 | LL: 1719 | dihydrofolate reductase |
| 26 | 221677_s_at | AF232674.1 | LL: 29980 | downstream neighbor of SON |
| 27 | 201479_at | NM_001363.1 | LL: 1736 | dyskeratosis congenita 1, dyskerin |
| 28 | 203358_s_at | NM_004456.1 | LL: 2146 | enhancer of zeste homolog 2 (Drosophila) |
| 29 | 204603_at | NM_003686.1 | LL: 9156 | exonuclease 1 |
| 30 | 204817_at | NM_012291.1 | LL: 9700 | extra spindle poles like 1 (S. cerevisiae) |
| 31 | 218875_s_at | NM_012177.1 | LL: 26271 | F-box only protein 5 |
| 32 | 204768_s_at | NM_004111.3 | LL: 2237 | flap structure-specific endonuclease 1 |
| 33 | 202580_x_at | NM_021953.1 | LL: 2305 | forkhead box M1 |
| 34 | 214804_at | BF793446 | LL: 2491 | FSH primary response (LRPR1 homolog, rat) 1 |
| 35 | 215942_s_at | BF973178 | LL: 51512 | G-2 and S-phase expressed 1 |
| 36 | 203560_at | NM_003878.1 | LL: 8836 | gamma-glutamyl hydrolase (conjugase, |
| | | | | folylpolygammaglutamyl hydrolase) |
| 37 | 205436_s_at | NM_002105.1 | LL: 3014 | H2A histone family, member X |
| 38 | 200853_at | NM_002106.1 | LL: 3015 | H2A histone family, member Z |
| 39 | 204162_at | NM_006101.1 | LL: 10403 | highly expressed in cancer, rich in leucine |
| | | | | heptad repeats |
| 40 | 208808_s_at | BC000903.1 | LL: 3148 | high-mobility group (nonhistone chromosomal) |
| | | | | protein 2 |
| 41 | 201292_at | NM_001067.1 | LL: 7153 | Homo sapiens (cell line HL-60) alpha topoisomerase |
| | | | | truncated-form mRNA, 3′UTR. |
| 42 | 221505_at | AW612574 | TSR: 311213 | Homo sapiens cDNA: FLJ21971 fis, clone HEP05790. |
| 43 | 222039_at | AA292789 | TSR: 46324 | Homo sapiens mRNA; cDNA DKFZp434N144 (from clone |
| | | | | DKFZp434N144). |
| 44 | 207165_at | NM_012485.1 | LL: 3161 | hyaluronan-mediated motility receptor (RHAMM) |
| 45 | 202854_at | NM_000194.1 | LL: 3251 | hypoxanthine phosphoribosyltransferase 1 (Lesch- |
| | | | | Nyhan syndrome) |
| 46 | 201088_at | NM_002266.1 | LL: 3838 | karyopherin alpha 2 (RAG cohort 1, importin alpha 1) |
| 47 | 218355_at | NM_012310.2 | LL: 24137 | kinesin family member 4A |
| 48 | 204444_at | NM_004523.2 | LL: 3832 | kinesin-like 1 |
| 49 | 204709_s_at | NM_004856.3 | LL: 9493 | kinesin-like 5 (mitotic kinesin-like protein 1) |
| 50 | 209408_at | U63743.1 | LL: 11004 | kinesin-like 6 (mitotic centromere-associated kinesin) |
| 51 | 219306_at | NM_020242.1 | LL: 56992 | kinesin-like 7 |
| 52 | 203276_at | NM_005573.1 | LL: 4001 | lamin B1 |
| 53 | 208103_s_at | NM_030920.1 | LL: 81611 | lecuine-rich acidic protein-like protein |
| 54 | 205240_at | NM_013296.1 | LL: 29899 | LGN protein |
| 55 | 204825_at | NM_014791.1 | LL: 9833 | likely ortholog of maternal embryonic leucine |
| | | | | zipper kinase |
| 56 | 203362_s_at | NM_002358.2 | LL: 4085 | MAD2 mitotic arrest deficient-like 1 (yeast) |
| 57 | 220651_s_at | NM_018518.1 | LL: 55388 | MCM10 minichromosome maintenance deficient 10 |
| | | | | (S. cerevisiae) |
| 58 | 202107_s_at | NM_004526.1 | LL: 4171 | MCM2 minichromosome maintenance deficient 2, |
| | | | | mitotin (S. cerevisiae) |
| 59 | 201555_at | NM_002388.2 | LL: 4172 | MCM3 minichromosome maintenance deficient 3 |
| | | | | (S. cerevisiae) |
| 60 | 212141_at | X74794.1 | LL: 4173 | MCM4 minichromosome maintenance deficient 4 |
| | | | | (S. cerevisiae) |
| 61 | 201930_at | NM_005915.2 | LL: 4175 | MCM6 minichromosome maintenance deficient 6 |
| | | | | (MIS5 homolog, S. pombe) (S. cerevisiae) |
| 62 | 210983_s_at | AF279900.1 | LL: 4176 | MCM7 minichromosome maintenance deficient 7 |
| | | | | (S. cerevisiae) |
| 63 | 203931_s_at | NM_002949.1 | LL: 6182 | mitochondrial ribosomal protein L12 |
| 64 | 203145_at | NM_006461.1 | LL: 10615 | mitotic spindle coiled-coil related protein |
| 65 | 218499_at | NM_016542.1 | LL: 51765 | Mst3 and SOK1-related kinase |
| 66 | 204641_at | NM_002497.1 | LL: 4751 | NIMA (never in mitosis gene a)-related kinase 2 |
| 67 | 201970_s_at | NM_002482.1 | LL: 4678 | nuclear autoantigenic sperm protein (histone-binding) |
| 68 | 218039_at | NM_016359.1 | LL: 51203 | nucleolar protein ANKT |
| 69 | 221923_s_at | AA191576 | LL: 4869 | nucleophosmin (nucleolar phosphoprotein B23, numatrin) |
| 70 | 213599_at | BE045993 | LL: 11339 | Opa-interacting protein 5 |
| 71 | 203554_x_at | NM_004219.2 | LL: 9232 | pituitary tumor-transforming 1 |
| 72 | 208511_at | NM_021000.1 | LL: 26255 | pituitary tumor-transforming 3 |
| 73 | 202240_at | NM_005030.1 | LL: 5347 | polo-like kinase (Drosophila) |
| 74 | 213226_at | AI346350 | LL: 5393 | polymyositis/scleroderma autoantigen 1 (75 kD) |
| 75 | 201202_at | NM_002592.1 | LL: 5111 | proliferating cell nuclear antigen |
| 76 | 218009_s_at | NM_003981.1 | LL: 9055 | protein regulator of cytokinesis 1 |
| 77 | 218755_at | NM_005733.1 | LL: 10112 | RAB6 interacting, kinesin-like (rabkinesin6) |
| 78 | 222077_s_at | AU153848 | LL: 29127 | Rac GTPase activating protein 1 |
| 79 | 205024_s_at | NM_002875.1 | LL: 5888 | RAD51 homolog (RecA homolog, E. coli) (S. cerevisiae) |
| 80 | 204146_at | BE966146 | LL: 10635 | RAD51-interacting protein |
| 81 | 202483_s_at | NM_002882.2 | LL: 5902 | RAN binding protein 1 |
| 82 | 218585_s_at | NM_016448.1 | LL: 51514 | RA-regulated nuclear matrix-associated protein |
| 83 | 204127_at | BC000149.2 | LL: 5983 | replication factor C (activator 1) 3 (38 kD) |
| 84 | 204023_at | NM_002916.1 | LL: 5984 | replication factor C (activator 1) 4 (37 kD) |
| 85 | 203022_at | NM_006397.1 | LL: 10535 | ribonuclease HI, large subunit |
| 86 | 201890_at | NM_001034.1 | LL: 6241 | ribonucleotide reductase M2 polypeptide |
| 87 | 209464_at | AB011446.1 | LL: 9212 | serine/threonine kinase 12 |
| 88 | 204092_s_at | NM_003600.1 | LL: 8465 | serine/threonine kinase 15 |
| 89 | 204887_s_at | NM_014264.1 | LL: 10733 | serine/threonine kinase 18 |
| 90 | 208079_s_at | NM_003158.1 | LL: 6790 | serine/threonine kinase 6 |
| 91 | 210691_s_at | AF275803.1 | LL: 27101 | Siah-interacting protein |
| 92 | 205644_s_at | NM_003096.1 | LL: 6637 | small nuclear ribonucleoprotein polypeptide G |
| 93 | 201664_at | AL136877.1 | LL: 10051 | SMC4 structural maintenance of chromosomes 4-like 1 |
| | | | | (yeast) |
| 94 | 209680_s_at | BC000712.1 | LL: 8831 | synaptic Ras GTPase activating protein 1 |
| | | | | homolog (rat) |
| 95 | 205339_at | NM_003035.1 | LL: 6491 | TAL1 (SCL) interrupting locus |
| 96 | 202589_at | NM_001071.1 | LL: 7298 | thymidylate synthetase |
| 97 | 203432_at | AW272611 | LL: 7112 | thymopoietin |
| 98 | 204033_at | NM_004237.1 | LL: 9319 | thyroid hormone receptor interactor 13 |
| 99 | 219148_at | NM_018492.1 | LL: 55872 | T-LAK cell-originated protein kinase |
| 100 | 201291_s_at | NM_001067.1 | LL: 7153 | topoisomerase (DNA) II alpha (170 kD) |
| 101 | 218308_at | NM_006342.1 | LL: 10460 | transforming, acidic coiled-coil containing |
| | | | | protein 3 |
| 102 | 204822_at | NM_003318.1 | LL: 7272 | TTK protein kinase |
| 103 | 202779_s_at | NM_014501.1 | LL: 27338 | ubiquitin carrier protein |
| 104 | 202413_s_at | NM_003368.1 | LL: 7398 | ubiquitin specific protease 1 |
| 105 | 202954_at | NM_007019.1 | LL: 11065 | ubiquitin-conjugating enzyme E2C |
| 106 | 219555_s_at | NM_018455.1 | LL: 55839 | uncharacterized bone marrow protein BM039 |
| 107 | 213906_at | AW592266 | LL: 4603 | v-myb myeloblastosis viral oncogenehomolog |
| | | | | (avian)-like 1 |
| 108 | 204026_s_at | NM_007057.1 | LL: 11130 | ZW10 interactor |

TABLE 2
Genes That Negatively Correlate With KSP Expression

| Differential | | GenBank | | | |
|---|---|---|---|---|---|
| Gene No. | Clone_ID | Accession No. | Locus Link | ALIAS | NAME |
| 109 | 204894_s_at | NM_003734.2 | LL: 8639 | AOC3 | amine oxidase, copper containing |
| | | | | | 3 (vascular adhesion protein 1) |
| 110 | 202920_at | BF726212 | LL: 287 | ANK2 | ankyrin 2, neuronal |
| 111 | 209047_at | AL518391 | LL: 358 | AQP1 | aquaporin 1 (channel-forming integral |
| | | | | | protein, 28 kD) |
| 112 | 204719_at | NM_007168.1 | LL: 10351 | ABCA8 | ATP-binding cassette, sub-family A |
| | | | | | (ABC1), member 8 |
| 113 | 211062_s_at | BC006393.1 | LL: 8532 | CPZ | carboxypeptidase Z |
| 114 | 212097_at | AU147399 | LL: 857 | CAV1 | caveolin 1, caveolae protein, 22 kD |
| 115 | 209543_s_at | M81104.1 | LL: 947 | CD34 | CD34 antigen |
| 116 | 206932_at | NM_003956.1 | LL: 9023 | CH25H | cholesterol 25-hydroxylase |
| 117 | 222043_at | AI982754 | LL: 1191 | CLU | clusterin (complement lysis inhibitor, |
| | | | | | SP-40, 40, sulfated glycoprotein 2, |
| | | | | | testosterone- repressed prostate |
| | | | | | message 2, apolipoprotein J) |
| 118 | 203305_at | NM_000129.2 | LL: 2162 | F13A1 | coagulation factor XIII, A1 polypeptide |
| 119 | 212865_s_at | BF449063 | LL: 7373 | COL14A1 | collagen, type XIV, alpha 1 (undulin) |
| 120 | 204345_at | NM_001856.1 | LL: 1307 | COL16A1 | collagen, type XVI, alpha 1 |
| 121 | 202992_at | NM_000587.1 | LL: 730 | C7 | complement component 7 |
| 122 | 204570_at | NM_001864.1 | LL: 1346 | COX7A1 | cytochrome c oxidase subunit VIIa |
| | | | | | polypeptide 1 (muscle) |
| 123 | 213661_at | AI671186 | LL: 25891 | DKFZP586H2123 | DKFZP586H2123 protein |
| 124 | 201041_s_at | NM_004417.2 | LL: 1843 | DUSP1 | dual specificity phosphatase 1 |
| 125 | 208335_s_at | NM_002036.1 | LL: 2532 | FY | Duffy blood group |
| 126 | 206580_s_at | NM_016938.1 | LL: 30008 | EFEMP2 | EGF-containing fibulin-like extracellular |
| | | | | | matrix protein 2 |
| 127 | 219436_s_at | NM_016242.1 | LL: 51705 | LOC51705 | endomucin-2 |
| 128 | 202768_at | NM_006732.1 | LL: 2354 | FOSB | FBJ murine osteosarcoma viral oncogene |
| | | | | | homolog B |
| 129 | 204359_at | NM_013231.1 | LL: 23768 | FLRT2 | fibronectin leucine rich transmembrane |
| | | | | | protein 2 |
| 130 | 201540_at | NM_001449.1 | LL: 2273 | FHL1 | four and a half LIM domains 1 |
| 131 | 203697_at | U91903.1 | LL: 2487 | FRZB | frizzled-related protein |
| 132 | 205384_at | NM_005031.2 | LL: 5348 | FXYD1 | FXYD domain containing ion transport |
| | | | | | regulator 1 (phospholemman) |
| 133 | 202177_at | NM_000820.1 | LL: 2621 | GAS6 | growth arrest-specific 6 |
| 134 | 207704_s_at | NM_003644.1 | LL: 8522 | GAS7 | growth arrest-specific 7 |
| 135 | 221447_s_at | NM_031302.1 | LL: 83468 | LOC83468 | gycosyltransferase |
| 136 | 213800_at | X04697.1 | LL: 3075 | HF1 | H factor 1 (complement) |
| 137 | 216866_s_at | M64108.1 | TSR: 37632 | 0 | Human udulin 1 mRNA, 3′ end. |
| 138 | 209541_at | NM_000618.1 | LL: 3479 | IGF1 | insulin-like growth factor 1 |
| | | | | | (somatomedin C) |
| 139 | 216331_at | AK022548.1 | LL: 3679 | ITGA7 | integrin, alpha 7 |
| 140 | 214927_at | AL359052.1 | LL: 9358 | ITGBL1 | integrin, beta-like 1 (with EGF-like |
| | | | | | repeat domains) |
| 141 | 205116_at | NM_000426.1 | LL: 3908 | LAMA2 | laminin, alpha 2 (merosin, congenital |
| | | | | | muscular dystrophy) |
| 142 | 203766_s_at | NM_012134.1 | LL: 25802 | LMOD1 | leiomodin 1 (smooth muscle) |
| 143 | 200785_s_at | NM_002332.1 | LL: 4035 | LRP1 | low density lipoprotein-related |
| | | | | | protein 1 (alpha-2-macroglobulin |
| | | | | | receptor) |
| 144 | 210794_s_at | AF119863.1 | LL: 55384 | MEG3 | maternally expressed 3 |
| 145 | 202350_s_at | NM_002380.2 | LL: 4147 | MATN2 | matrilin 2 |
| 146 | 207118_s_at | NM_004659.1 | LL: 8511 | MMP23A | matrix metalloproteinase 23A |
| 147 | 212713_at | R72286 | LL: 4239 | MFAP4 | microfibrillar-associated protein 4 |
| 148 | 207961_x_at | NM_022870.1 | LL: 4629 | MYH11 | myosin, heavy polypeptide 11, smooth |
| | | | | | muscle |
| 149 | 202555_s_at | NM_005965.1 | LL: 4638 | MYLK | myosin, light polypeptide kinase |
| 150 | 209550_at | U35139.1 | LL: 4692 | NDN | necdin homolog (mouse) |
| 151 | 218730_s_at | NM_014057.1 | LL: 4969 | OGN | osteoglycin (osteoinductive factor, |
| | | | | | mimecan) |
| 152 | 219628_at | NM_022470.1 | LL: 64393 | WIG1 | p53 target zinc finger protein |
| 153 | 219132_at | NM_021255.1 | LL: 57161 | PELI2 | pellino homolog 2 (Drosophila) |
| 154 | 208396_s_at | NM_005019.1 | LL: 5136 | PDE1A | phosphodiesterase 1A, calmodulin- |
| | | | | | dependent |
| 155 | 204134_at | NM_002599.1 | LL: 5138 | PDE2A | phosphodiesterase 2A, cGMP-stimulated |
| 156 | 210831_s_at | L27489.1 | LL: 5733 | PTGER3 | prostaglandin E receptor 3 (subtype EP3) |
| 157 | 207177_at | NM_000959.1 | LL: 5737 | PTGFR | prostaglandin F receptor (FP) |
| 158 | 206049_at | NM_003005.2 | LL: 6403 | SELP | selectin P (granule membrane protein |
| | | | | | 140 kD, antigen CD62) |
| 159 | 205405_at | NM_003966.1 | LL: 9037 | SEMA5A | sema domain, seven thrombospondin |
| | | | | | repeats (type 1 and type 1-like), |
| | | | | | transmembrane domain (TM) and short |
| | | | | | cytoplasmic domain, (semaphorin) 5A |
| 160 | 209897_s_at | AF055585.1 | LL: 9353 | SLIT2 | slit homolog 2 (Drosophila) |
| 161 | 203812_at | AB011538.1 | LL: 6586 | SLIT3 | slit homolog 3 (Drosophila) |
| 162 | 205392_s_at | NM_004166.1 | LL: 6358 | SCYA14 | small inducible cytokine subfamily A |
| | | | | | (Cys—Cys), member 14 |
| 163 | 200795_at | NM_004684.1 | LL: 8404 | SPARCL1 | SPARC-like 1 (mast9, hevin) |
| 164 | 206093_x_at | NM_007116.1 | LL: 7148 | TNXB | tenascin XB |
| 165 | 209747_at | J03241.1 | LL: 7043 | TGFB3 | transforming growth factor, beta 3 |
| 166 | 208944_at | D50683.1 | LL: 7048 | TGFBR2 | transforming growth factor, beta |
| | | | | | receptor II (70-80 kD) |
| 167 | 202242_at | NM_004615.1 | LL: 7102 | TM4SF2 | transmembrane 4 superfamily member 2 |
| 168 | 213541_s_at | AI351043 | LL: 2078 | ERG | v-ets erythroblastosis virus E26 |
| | | | | | oncogene like (avian) |
| 169 | 202112_at | NM_000552.2 | LL: 7450 | VWF | von Willebrand factor |

TABLE 3
Genes From Table 1 that Show Strongest Positive Correlation with KSP

| Fragment | | | Locus Link | |
|---|---|---|---|---|
| Name | Gene Name | Genbank ID | ID | Function |
| 202095_s_at | baculoviral IAP repeat-containing | NM_001168 | LL: 332 | GO:0008189:apoptosis inhibitor |
| | 5 (survivin) | | | |
| 209642_at | BUB1 budding uninhibited by | AF043294 | LL: 699 | GO:0004672:protein kinase |
| | benzimidazoles 1 homolog (yeast) | | | |
| 203213_at | cell division cycle 2, G1 to S | AL524035 | LL: 983 | GO:0004672:protein kinase, |
| | and G2 to M | | | GO:0004693:cyclin-dependent |
| | | | | protein kinase |
| 205046_at | centromere protein E (312 kD) | NM_001813 | LL: 1062 | GO:0008350:kinetochore motor |
| 210052_s_at | chromosome 20 open reading frame 1 | AF098158 | LL: 22974 | GO:0005524:ATP binding, |
| | | | | GO:0005525:GTP binding |
| 218662_s_at | chromosome condensation protein G | NM_022346 | LL: 64151 | 0 |
| 214710_s_at | cyclin B1 | BE407516 | LL: 891 | 0 |
| 202580_x_at | forkhead box M1 | NM_021953 | LL: 2305 | GO:0003677:DNA binding, |
| | | | | GO:0003700:transcription factor, |
| | | | | GO:0003702:RNA polymerase II |
| | | | | transcription factor |
| 201292_at | Homo sapiens (cell line HL-60) alpha | AL561834 | TSR: 72473 | 0 |
| | topoisomerase truncated-form mRNA, | | | |
| | 3′UTR. | | | |
| 222039_at | Homo sapiens mRNA; cDNA DKFZp434N144 | AA292789 | TSR: 46324 | 0 |
| | (from clone DKFZp434N144). | | | |
| 207165_at | hyaluronan-mediated motility | NM_012485 | LL: 3161 | GO:0005540:hyaluronic acid binding |
| | receptor (RHAMM) | | | |
| 219787_s_at | hypothetical protein FLJ10461 | NM_018098 | LL: 55710 | 0 |
| 221520_s_at | hypothetical protein FLJ10468 | BC001651 | LL: 55143 | 0 |
| 219918_s_at | hypothetical protein FLJ10517 | NM_018123 | LL: 55158 | 0 |
| 202503_s_at | KIAA0101 gene product | NM_014736 | LL: 9768 | 0 |
| 206102_at | KIAA0186 gene product | NM_021067 | LL: 9837 | 0 |
| 204444_at | kinesin-like 1 | NM_004523 | LL: 3832 | GO:0003777:microtubule motor, |
| | | | | GO:0004002:adenosinetriphosphatase |
| 209408_at | kinesin-like 6 (mitotic | U63743 | LL: 11004 | GO:0003777:microtubule motor |
| | centromere-associated kinesin) | | | |
| 203362_s_at | MAD2 mitotic arrest deficient-like | NM_002358 | LL: 4085 | 0 |
| | 1 (yeast) | | | |
| 222036_s_at | MCM4 minichromosome maintenance | AI859865 | LL: 4173 | GO:0003677:DNA binding, |
| | deficient 4 (S. cerevisiae) | | | GO:0004002:adenosinetriphosphatase |
| 204641_at | NIMA (never in mitosis gene a)- | NM_002497 | LL: 4751 | GO:0004674:protein serine/threonine |
| | related kinase 2 | | | kinase |
| 218039_at | nucleolar protein ANKT | NM_016359 | LL: 51203 | 0 |
| 203554_x_at | pituitary tumor-transforming 1 | NM_004219 | LL: 9232 | GO:0003700:transcription factor |
| 213226_at | polymyositis/scleroderma autoantigen | AI346350 | LL: 5393 | 0 |
| | 1 (75 kD) | | | |
| 218782_s_at | PRO2000 protein | NM_014109 | LL: 29028 | 0 |
| 218009_s_at | protein regulator of cytokinesis 1 | NM_003981 | LL: 9055 | 0 |
| 222077_s_at | Rac GTPase activating protein 1 | AU153848 | LL: 29127 | 0 |
| 204146_at | RAD51-interacting protein | BE966146 | LL: 10635 | GO:0003723:RNA binding, |
| | | | | GO:0003690:double-stranded DNA binding, |
| | | | | GO:0003697:single-stranded DNA binding |
| 203209_at | replication factor C (activator 1) | BC001866 | LL: 5985 | 0 |
| | 5 (36.5 kD) | | | |
| 209773_s_at | ribonucleotide reductase M2 | BC001886 | LL: 6241 | 0 |
| | polypeptide | | | |
| 204092_s_at | serine/threonine kinase 15 | NM_003600 | LL: 8465 | GO:0004672:protein kinase |
| 219148_at | T-LAK cell-originated protein kinase | NM_018492 | LL: 55872 | 0 |
| 204822_at | TTK protein kinase | NM_003318 | LL: 7272 | GO:0004713:protein tyrosine kinase, |
| | | | | GO:0004674:protein serine/threonine |
| | | | | kinase |
| 204026_s_at | ZW10 interactor | NM_007057 | LL: 11130 | 0 |

TABLE 4
Genes from Table 2 that Show Strongest Negative Correlation with KSP

| Fragment | | | | |
|---|---|---|---|---|
| Name | Gene Name | Gen Bank Acc | Locus Link | Function |
| 211986_at | AHNAK nucleoprotein (desmoyokin) | BG287862 | LL: 195 | 0 |
| 204719_at | ATP-binding cassette, sub-family | NM_007168 | LL: 10351 | 0 |
| | A (ABC1), member 8 | | | |
| 204167_at | biotinidase | NM_000060 | LL: 686 | GO:0004075:biotin carboxylase |
| 204581_at | CD22 antigen | NM_001771 | LL: 933 | GO:0005194:cell adhesion |
| 204570_at | cytochrome c oxidase subunit VIIa | NM_001864 | LL: 1346 | GO:0004129:cytochrome-c oxidase |
| | polypeptide 1 (muscle) | | | |
| 218418_s_at | DKFZP434N161 protein | NM_015493 | LL: 25959 | 0 |
| 214919_s_at | eukaryotic translation initiation | R39094 | LL: 8637 | 0 |
| | factor 4E binding protein 3 | | | |
| 205384_at | FXYD domain containing ion | NM_005031 | LL: 5348 | GO:0005254:chloride channel |
| | transport regulator 1 | | | |
| | (phospholemman) | | | |
| 219747_at | hypothetical protein FLJ23191 | NM_024574 | LL: 79625 | 0 |
| 201508_at | insulin-like growth factor | NM_001552 | LL: 3487 | GO:0005067:insulin-like growth |
| | binding protein 4 | | | factor receptor binding protein |
| 209002_s_at | KIAA1536 protein | BC003177 | LL: 57658 | 0 |
| 216264_s_at | laminin, beta 2 (laminin S) | X79683 | LL: 3913 | GO:0005198:structural protein |
| 220392_at | likely ortholog of mouse early | NM_022659 | LL: 64641 | 0 |
| | B-cell factor 2 | | | |
| 222161_at | N-acetylated alpha-linked acidic | AJ012370 | LL: 10003 | GO:0008239:dipeptidyl-peptidase |
| | dipeptidase 2 | | | |
| 210249_s_at | nuclear receptor coactivator 1 | U59302 | LL: 8648 | GO:0003713:transcription co-activator |
| 208522_s_at | patched homolog (Drosophila) | NM_000264 | LL: 5727 | GO:0004872:receptor, |
| | | | | GO:0008181:tumor suppressor |
| 36829_at | period homolog 1 (Drosophila) | AF022991 | LL: 5187 | 0 |
| 206380_s_at | properdin P factor, complement | NM_002621 | LL: 5199 | GO:0005211:plasma glycoprotein, |
| | | | | GO:0003811 complement component, |
| | | | | GO:0003797:antibacterial response |
| | | | | protein |
| 216300_x_at | retinoic acid receptor, alpha | BE383139 | LL: 5914 | GO:0003700:transcription factor, |
| | | | | GO:0003708:retinoic acid receptor, |
| | | | | GO:0003713:transcription co- |
| | | | | activator |
| 204906_at | ribosomal protein S6 kinase, | BC002363 | LL: 6196 | GO:0004674:protein serine/threonine |
| | 90 kD, polypeptide 2 | | | kinase |
| 205392_s_at | small inducible cytokine subfamily | NM_004166 | LL: 6358 | GO:0004871:signal transduction |
| | A (Cys—Cys), member 14 | | | |
| 206093_x_at | tenascin XB | NM_007116 | LL: 7148 | 0 |
| 207134_x_at | tryptase beta 1 | NM_024164 | LL: 7177 | GO:0008236:serine-type peptidase |
| 217023_x_at | tryptase beta 2 | AF099143 | LL: 64499 | 0 |
| 210084_x_at | tryptase, alpha | AF206665 | LL: 7176 | GO:0008236:serine-type peptidase |
| 205883_at | zinc finger protein 145 | NM_006006 | LL: 7704 | GO:0005515:protein binding, |
| | (Kruppel-like, expressed in | | | GO:0003700:transcription factor, |
| | promyelocytic leukemia) | | | GO:0003714:transcription co-repressor, |
| | | | | GO:0016251:general RNA polymerase II |
| | | | | transcription factor |

What is claimed is:

1. A method of classifying a tumor, comprising:
(a) providing a test sample derived from a tumor cell, wherein the tumor cell is capable of expressing one or more nucleic acid markers selected from the group consisting of those listed in Table 1 and Table 2;
(b) determining the expression level of the one or more nucleic acid markers in the test sample;
(c) comparing the expression level of the one or more nucleic acid markers in the test sample with the expression level of the one or more nucleic acid markers in a control sample whose tumor status is known; and
(d) classifying the tumor cell on the basis of the comparison of step (c).

2. The method of claim 1, wherein the control sample is representative of a known tumor.

3. The method of claim 1, wherein the tumor is a cancer.

4. The method of claim 1, wherein the expression level of at least five of the nucleic acid markers is determined.

5. The method of claim 4, wherein the expression level of at least ten of the nucleic acid markers is determined.

6. The method of claim 5, wherein the expression level of at least twenty-five of the nucleic acid markers is determined.

7. The method of claim 1, wherein the list of nucleic acid markers is selected from the group consisting of those listed in Table 3 or Table 4.

8. The method of claim 1, wherein the tumor cell is from breast, ovary, or lung tissue.

9. The method of claim 1, wherein the expression levels of the one or more nucleic acids is compared with the expression level of the same nucleic acid markers from control samples representative of a plurality of known tumors.

10. The method of claim 2, wherein determination of expression levels comprises determining the transcript levels for the one or more nucleic acid markers.

11. The method of claim 1, wherein determination of expression levels comprises determining the protein levels for the one or more nucleic acid markers.

12. The method of claim 1, wherein determining is performed by probe array analysis.

13. The method of claim 1, wherein determining is performed by a quantitative PCR method.

14. The method of claim 1, wherein the tumor cell is obtained from a mammal.

15. The method of claim 14, wherein the tumor cell is obtained from a human.

16. The method of claim 1, wherein the test sample is provided in vitro.

17. The method of claim 1, wherein the test sample is provided ex vivo.

18. The method of claim 1, wherein the test sample is provided in vivo.

19. The method of claim 1, wherein the one or more nucleic acids include at least one nucleic acid from each of Table 1 and Table 2; and wherein
if the expression levels of one or more of the nucleic acids from Table 1 are increased and one or more of the nucleic acids from Table 2 are decreased relative to the corresponding expression levels in the control sample, then the test sample is classified as one having a high mitotic index; and
if the expression levels of one or more of the nucleic acids from Table 1 are decreased and one or more of the nucleic acids from Table 2 are increased relative to the corresponding expression levels in the control sample, then the test sample is classified as having a low mitotic index.

20. A method of determining whether a cancerous tissue is treatable with an inhibitor of KSP, comprising:
(a) providing a test sample derived from a cancerous tissue from a subject;
(b) determining the expression levels of one or more markers from Table 1 and Table 2 in the cancerous tissue, wherein an increase in expression of one or more markers from those listed in Table 1 and a decrease in expression of one or more markers from those listed in Table 2 relative to the levels of these markers in a normal sample of the same type of tissue indicates that the cancerous tissue is treatable by the inhibitor of KSP.

21. The method of claim 20, wherein the inhibitor is a quinazolinone derivative.

22. The method of claim 20, wherein the expression levels of at least five markers from each of Table I and Table II are determined.

23. The method of claim 22, wherein the expression levels of at least ten markers from each of Table I and Table II are determined.

24. The method of claim 23, wherein the expression levels of at least twenty-five markers from each of Table I and Table II are determined.

25. The method of claim 20, wherein the cancerous tissue is obtained from breast, ovary or lung tissue.

26. The method of claim 20, wherein determination of expression levels comprises determining the transcript levels for the one or more nucleic acid markers.

27. The method of claim 20, wherein determination of expression levels comprises determining the protein levels for the one or more nucleic acid markers.

28. The method of claim 20, wherein the subject is a mammal.

29. The method of claim 28, wherein the subject is a human.

30. A method for diagnosing the presence of, or predisposition to, a tumor in a subject, comprising:
(a) determining the expression level of one or more nucleic acid markers in a test sample obtained from the subject, wherein the one or more nucleic acid markers are selected from the group consisting of those listed in Table 1 and Table 2;
(b) comparing the expression level of the one or more nucleic acid markers in the test sample with the expression level of these same nucleic acid markers in a control sample whose tumor status is known; and
(c) diagnosing the presence or absence of the tumor in the subject, or a predisposition to the tumor by the subject, on the basis of the comparison of step (b).

31. The method of claim 30, wherein the expression level of at least five of the nucleic acid markers is determined.

32. The method of claim 31, wherein the expression level of at least ten of the nucleic acid markers is determined.

33. The method of claim 32, wherein the expression level of at least twenty-five of the nucleic acid markers is determined.

34. The method of claim 30, wherein the list of nucleic acid markers is selected from the group of those listed in Table 3 and Table 4.

35. The method of claim 30, wherein the known cancer or tumor is a breast cancer, ovarian cancer or lung cancer.

36. The method of claim 30, wherein
the control sample is representative of an individual or population not having the cancer or tumor; and
the diagnosing step comprises diagnosing the presence of a tumor if the expression levels of the one or more nucleic acids differs from the corresponding expression levels in the control sample.

37. The method of claim 30, wherein determination of expression levels comprises determining transcript levels for the one or more nucleic acid markers.

38. The method of claim 30, wherein determination of expression levels comprises determining protein levels for those proteins encoded by the one or more nucleic acid markers.

39. The method of claim 30, wherein the subject is a mammal.

40. The method of claim 39, wherein the subject is a human.

41. A screening method to identify an inhibitor of a tumor, the method comprising:
(a) contacting a test cell capable of expressing one or more nucleic acid markers selected from the group comprising those listed in Table 1 or Table 2 with a test agent;
(b) determining the expression level of one or more nucleic acid markers comprising those listed in Table 1 and Table 2;
(c) comparing the expression level of the one or more nucleic acid markers with the expression level of the same markers for a control cell population whose tumor status is known and that has not been contacted with the test agent; and
(d) identifying the test agent as an inhibitor of the tumor on the basis of the comparison step (c).

42. A method for assessing whether a test agent is a potential carcinogen, the method comprising:
(a) contacting a test cell capable of expressing one or more nucleic acid markers selected from the group consisting of those listed in Table 1 or Table 2 with the test agent;
(b) determining the expression level of one or more nucleic acid markers selected from the group of those listed in Table 1 and Table 2;
(c) comparing the expression level of the one or more nucleic acid markers with the expression level of the same markers for a control cell population that is representative of cells from tissue having the cancer and/or not having the cancer; and
(d) identifying a test agent as a potential carcinogen or not on the basis of the comparison step (c).

43. A method of treating a tumor with a high mitotic index, comprising administering to a subject having the tumor, or at risk of developing the tumor, a pharmaceutical agent that inhibits the expression or activity of one or more nucleic acid markers selected from the group consisting of those listed in Table 1 and/or activates the expression or activity of one or more nucleic acids selected from the group consisting of those listed in Table 2.

44. The method of claim 43, wherein the tumor is present in the breast, ovary or lung of the subject.

45. The method of claim 43, wherein the pharmaceutical agents is a KSP inhibitor.

46. A method of treating a tumor with a low mitotic index, comprising administering to a subject having the tumor, or at risk of developing the tumor, a pharmaceutical agent that activates the expression or activity of one or more nucleic acid markers selected from the group consisting of those listed in Table 1 and/or inhibits the expression or activity of one or more nucleic acids selected from the group consisting of those listed in Table 2.

47. The method of claim 46, wherein the tumor is present in the breast, ovary or lung of the subject.
